(19) United States
(12) Patent Application Publication
(10) Pub. No.: US 2026/0079126 A1
(43) Pub. Date: Mar. 19, 2026

(54) GAS SENSOR

(71) Applicant: NGK INSULATORS, LTD., Nagoya-shi (JP)

(72) Inventor: Taku OKAMOTO, Nagoya-shi (JP)

(21) Appl. No.: 19/346,620

(22) Filed: Oct. 1, 2025

Related U.S. Application Data
(63) Continuation of application No. PCT/JP2024/001720, filed on Jan. 22, 2024.

(30) Foreign Application Priority Data
May 9, 2023 (JP) 2023-077521

(51) Int. Cl. G01N 27/407; G01N 27/41; G01N 33/00

(52) U.S. Cl. CPC G01N 27/4071; G01N 27/41; G01N 33/0027

(57) ABSTRACT

A gas sensor includes a sensor element and a control unit for controlling the sensor element. The sensor element includes: a base part; a reference gas chamber formed between a proton-conductive solid electrolyte layer and an oxygen-ion-conductive solid electrolyte layer inside the base part, into the reference gas chamber an outside gas being introduced via an outside gas diffusion-rate limiting path; a hydrogen reference electrode disposed on the proton-conductive solid electrolyte layer in the reference gas chamber; a hydrogen generation pump cell including a hydrogen generation electrode disposed on the oxygen-ion-conductive solid electrolyte layer in the reference gas chamber; and a detection electrode disposed on the proton-conductive solid electrolyte layer to be in contact with a measurement-object gas. The control unit includes: a reference gas adjusting part for adjusting a hydrogen concentration in the reference gas chamber by operating the hydrogen generation pump cell; and a detecting part.

### CROSS-REFERENCE TO RELATED APPLICATION

This application is a continuation application of PCT/JP2024/001720, filed on Jan. 22, 2024, which claims priority from Japanese Patent Application No. 2023-077521, filed on May 9, 2023, the entire contents of which are incorporated herein by reference.

### BACKGROUND OF THE INVENTION

### Technical Field of the Invention

The present invention relates to a gas sensor including a sensor element using an ion-conductive solid electrolyte.

### Background Art

Various measurement devices are used for measurement of concentration of an objective gas component in a mixed gas, and as an example, a gas sensor using a hydrogen ion (or, a proton) conductive solid electrolyte (namely, a proton conductor) is known. For example, the Non-Patent Document 1 and the Non-Patent Document 2 disclose a hydrogen sensor using a proton conductive solid electrolyte. The hydrogen sensor detects hydrogen by an electromotive force (EMF) between an electrode located on a surface of the proton conductive solid electrolyte in contact with a measurement gas and a reference electrode located on a surface of the proton conductive solid electrolyte in contact with a reference gas.

JP 6667192 B2 discloses an ammonia sensor element using a proton conducting solid electrolyte. In the ammonia sensor element, a reference electrode is formed facing a reference gas chamber and in contact with a reference gas.

For example, JP 7122935 B2 discloses a carbon dioxide detecting device provided with a sensor element and a control unit. As one embodiment of the sensor element, JP 7122935 B2 discloses a sensor element including an ion conductor that conducts oxygen ions, a proton conductor that conducts hydrogen protons, and a gas chamber formed between the ion conductor and the proton conductor. The sensor element has a water detection electrode formed on a surface of the proton conductor, and a reference electrode formed on a surface of the proton conductor opposite to the surface on which the water detection electrode is formed. The reference electrode is in contact with a reference gas.

JP 2022-110596 A discloses a water vapor sensor which has a joint surface where a proton conductive solid electrolyte layer and an oxide ion conductive solid electrolyte layer are joined, and does not need a standard gas (namely, a reference gas).

### CITATION LIST

### Patent Documents

Patent Document 1: JP 6667192 B2

Patent Document 2: JP 7122935 B2

Patent Document 3: JP 2022-110596 A

### Non-Patent Documents

Non-Patent Document 1: M. K. Hossain et al., Nanomaterials 2022, 12, 3581

Non-Patent Document 2: Y. Okuyama et al., RSC Advances 2016, 6, 34019-34026

### SUMMARY OF THE INVENTION

### Problems to be Solved by the Invention

A gas sensor using a proton conductor is normally provided with a reference electrode that serves as reference for measurement of hydrogen concentration. The reference electrode is in contact with a reference gas that serves as reference of hydrogen concentration. When a gas with a predetermined hydrogen concentration is used as the reference gas, a gas cylinder or the like is required to supply the reference gas, and the gas sensor thus becomes large. On the other hand, the Non-Patent Document 2, JP 6667192 B2, and JP 7122935 B2 disclose that the air is used for the reference gas. In this case, the gas sensor can be minimized. However, hydrogen concentration in the air is extremely low and not stable. In such a case, there is a concern that measurement accuracy of hydrogen gas, or a gas containing a hydrogen atom may be degraded in the gas sensor. Here, the gas containing the hydrogen atom include ammonia NH₃, water vapor H₂O, hydrocarbon HC, and the like. Examples of hydrocarbon HC include alkane such as methane CH₄, and alkene such as ethylene C₂H₄.

It is therefore an object of the present invention to provide a gas sensor that can measure hydrogen gas or a gas (such as ammonia NH₃, water vapor H₂O, and hydrocarbon HC) containing a hydrogen atom in a measurement-object gas with higher accuracy.

### Means for Solving the Problems

The present inventor has intensively studied and as a result has found that by generating hydrogen from water vapor in an outside gas by a hydrogen generation pump cell to make a reference gas containing hydrogen be present in a reference gas chamber, hydrogen gas or a gas (such as ammonia NH₃, water vapor H₂O, and hydrocarbon HC) containing a hydrogen atom in a measurement-object gas can be measured with higher accuracy.

The present invention includes the following aspects.

(1) A gas sensor for detecting a target gas to be measured in a measurement-object gas, the gas sensor comprising a sensor element and a control unit for controlling the sensor element, wherein
the sensor element comprises:
   a base part in an elongated plate shape, including a proton-conductive solid electrolyte layer and an oxygen-ion-conductive solid electrolyte layer;
   a reference gas chamber formed between the proton-conductive solid electrolyte layer and the oxygen-ion-conductive solid electrolyte layer inside the base part, into the reference gas chamber an outside gas being introduced via an outside gas diffusion-rate limiting path;
   a hydrogen reference electrode disposed on the proton-conductive solid electrolyte layer in the reference gas chamber;
   a hydrogen generation pump cell including: a hydrogen generation electrode disposed on the oxygen-ion-conductive solid electrolyte layer in the reference gas chamber; and an outer electrode disposed at a position different from the reference gas chamber on the oxygen-ion-conductive solid electrolyte layer and corresponding to the hydrogen generation electrode; and
   a detection electrode disposed on the proton-conductive solid electrolyte layer to be in contact with a measurement-object gas; and
the control unit comprises:
   a reference gas adjusting part for adjusting a hydrogen concentration in the reference gas chamber by operating the hydrogen generation pump cell; and
   a detecting part for detecting a target gas to be measured in a measurement-object gas.

(2) The gas sensor according to the above (1), wherein the reference gas adjusting part adjusts the hydrogen concentration in the reference gas chamber by applying a predetermined voltage between the hydrogen generation electrode and the outer electrode of the hydrogen generation pump cell to decompose water vapor in the outside gas introduced into the reference gas chamber at the hydrogen generation electrode so that hydrogen and oxygen are generated, and to pump out the generated oxygen and an oxygen contained in the outside gas from the reference gas chamber.

(3) The gas sensor according to the above (1) or (2), wherein the detecting part detects the target gas to be measured in the measurement-object gas based on an electromotive force between the detection electrode and the hydrogen reference electrode.

(4) The gas sensor according to any one of the above (1) to (3), wherein the sensor element further comprises a measurement-object gas cavity formed inside the base part, into the measurement-object gas cavity the measurement-object gas being introduced via a measurement-object gas diffusion-rate limiting path,
the detection electrode exits in the measurement-object gas cavity, and
the detecting part detects the target gas to be measured in the measurement-object gas based on a current flowing between the detection electrode and the hydrogen reference electrode.

Detection (or, concentration measurement) of the target gas to be measured in the measurement-object gas may be performed based on the electromotive force between the detection electrode and the hydrogen reference electrode as in the case of the above (3), or may be performed based on the current between the detection electrode and the hydrogen reference electrode as in the case of the above (4). Alternatively, it is considerable that detection based on the electromotive force of the above (3) and detection based on the current of the above (4) may be performed in parallel.

(5) The gas sensor according to any one of the above (1) to (4), wherein the sensor element comprises:
a pretreatment chamber formed between the proton-conductive solid electrolyte layer and the oxygen-ion-conductive solid electrolyte layer inside the base part and adjacent to the reference gas chamber via the outside gas diffusion-rate limiting path, into the pretreatment chamber the outside gas being introduced via a pretreatment diffusion-rate limiting path; and
an oxygen pump cell including: an oxygen pump electrode disposed on the oxygen-ion-conductive solid electrolyte layer in the pretreatment chamber; and an outer electrode disposed at a position different from the reference gas chamber and the pretreatment chamber on the oxygen-ion-conductive solid electrolyte layer and corresponding to the oxygen pump electrode, and
the reference gas adjusting part adjusts the hydrogen concentration in the reference gas chamber by operating the oxygen pump cell to pump out oxygen in the outside gas introduced into the pretreatment chamber; and operating the hydrogen generation pump cell to decompose water vapor in the outside gas introduced into the reference gas chamber after the oxygen in the outside gas is pumped out in the pretreatment chamber so that hydrogen and oxygen are generated, and to pump out the generated oxygen and the oxygen contained in the outside gas from the reference gas chamber.

(6) The gas sensor according to any one of the above (1) to (5), wherein the sensor element further comprises an oxygen reference electrode disposed on the oxygen-ion-conductive solid electrolyte layer in the reference gas chamber, and
the reference gas adjusting part operates the hydrogen generation pump cell based on an electromotive force between the hydrogen reference electrode and the oxygen reference electrode.

(7) The gas sensor according to any one of the above (1) to (6), wherein the outer electrode of the hydrogen generation pump cell is disposed to be in contact with the measurement-object gas.

(8) The gas sensor according to any one of the above (1) to (7), wherein the target gas to be measured is hydrogen, ammonia, water vapor, or methane.

(9) A sensor element for detecting a target gas to be measured in a measurement-object gas, the sensor element comprising:
a base part in an elongated plate shape, including a proton-conductive solid electrolyte layer and an oxygen-ion-conductive solid electrolyte layer;
a reference gas chamber formed between the proton-conductive solid electrolyte layer and the oxygen-ion-conductive solid electrolyte layer inside the base part, into the reference gas chamber an outside gas being introduced via an outside gas diffusion-rate limiting path;
a hydrogen reference electrode disposed on the proton-conductive solid electrolyte layer in the reference gas chamber;
a hydrogen generation pump cell including: a hydrogen generation electrode disposed on the oxygen-ion-conductive solid electrolyte layer in the reference gas chamber; and an outer electrode disposed at a position different from the reference gas chamber on the oxygen-ion-conductive solid electrolyte layer and corresponding to the hydrogen generation electrode; and
a detection electrode disposed on the proton-conductive solid electrolyte layer to be in contact with a measurement-object gas.

(10) A gas adjusting device comprising:
a gas chamber at least partially surrounded by a proton-conductive solid electrolyte layer and an oxygen-ion-conductive solid electrolyte layer, into the gas chamber an outside gas being introduced via an outside gas diffusion-rate limiting path;
a hydrogen generation pump cell including: a hydrogen generation electrode disposed on the oxygen-ion-conductive solid electrolyte layer in the gas chamber; and an outer electrode disposed at a position different from the gas chamber on the oxygen-ion-conductive solid electrolyte layer and corresponding to the hydrogen generation electrode;
a hydrogen reference electrode disposed on the proton-conductive solid electrolyte layer in the gas chamber; and a gas adjusting part for adjusting a hydrogen concentration in the gas chamber by operating the hydrogen generation pump cell.

(11) A gas adjusting device comprising:
a gas chamber at least partially surrounded by a proton-conductive solid electrolyte layer and an oxygen-ion-conductive solid electrolyte layer, into the gas chamber an outside gas being introduced via an outside gas diffusion-rate limiting path;
a hydrogen generation pump cell including: a hydrogen generation electrode disposed on the oxygen-ion-conductive solid electrolyte layer in the gas chamber; and an outer electrode disposed at a position different from the gas chamber on the oxygen-ion-conductive solid electrolyte layer and corresponding to the hydrogen generation electrode;
a hydrogen reference electrode disposed on the proton-conductive solid electrolyte layer in the gas chamber; and
a gas adjusting part for adjusting a hydrogen concentration in the gas chamber by operating the hydrogen generation pump cell, wherein
the gas adjusting part adjusts the hydrogen concentration in the gas chamber by applying a predetermined voltage between the hydrogen generation electrode and the outer electrode of the hydrogen generation pump cell to decompose water vapor in the outside gas introduced into the gas chamber at the hydrogen generation electrode so that hydrogen and oxygen are generated, and to pump out the generated oxygen and an oxygen contained in the outside gas from the gas chamber; and supplies the gas whose hydrogen concentration is adjusted to the hydrogen reference electrode.

### Advantageous Effect of the Invention

According to the present invention, it is possible to provide a gas sensor that can measure hydrogen gas or a gas (such as ammonia NH₃, water vapor H₂O, and hydrocarbon HC) containing a hydrogen atom in a measurement-object gas with higher accuracy.

### BRIEF DESCRIPTION OF THE DRAWINGS

FIG. 1 is a vertical sectional schematic view in a longitudinal direction of a sensor element 101, showing one example of a schematic configuration of a gas sensor 100 of Embodiment 1.

FIG. 2 is a block diagram showing electric connections between a control unit 90, and respective cells 20 and 31 of the sensor element 101, in the gas sensor 100 of Embodiment 1.

FIG. 3 is a vertical sectional schematic view in a longitudinal direction of a sensor element 201, showing one example of a schematic configuration of a gas sensor 200 of Embodiment 2.

FIG. 4 is a block diagram showing electric connections between a control unit 290, and respective cells 21 and 31 of the sensor element 201, in the gas sensor 200 of Embodiment 2.

FIG. 5 is a vertical sectional schematic view in a longitudinal direction of a sensor element 301, showing one example of a schematic configuration of a gas sensor 300 of Embodiment 3.

FIG. 6 is a block diagram showing electric connections between a control unit 390, and respective cells 20, 31 and 51 of the sensor element 301, in the gas sensor 300 of Embodiment 3.

FIG. 7 is a vertical sectional schematic view in a longitudinal direction of a sensor element 401, showing one example of a schematic configuration of a gas sensor 400 of Embodiment 4.

FIG. 8 is a block diagram showing electric connections between a control unit 490, and respective cells 20, 31 and 61 of the sensor element 401, in the gas sensor 400 of Embodiment 4.

FIG. 9 is a vertical sectional schematic view in a longitudinal direction of a sensor element 501, showing one example of a schematic configuration of a gas sensor 500 of Embodiment 5.

### MODES FOR CARRYING OUT OF THE INVENTION

A gas sensor of the present invention includes a sensor element and a control unit for controlling the sensor element.

The sensor element contained in the gas sensor of the present invention includes:
a base part in an elongated plate shape, including a proton-conductive solid electrolyte layer and an oxygen-ion-conductive solid electrolyte layer;
a reference gas chamber formed between the proton-conductive solid electrolyte layer and the oxygen-ion-conductive solid electrolyte layer inside the base part, into the reference gas chamber an outside gas being introduced via an outside gas diffusion-rate limiting path;
a hydrogen reference electrode disposed on the proton-conductive solid electrolyte layer in the reference gas chamber;
a hydrogen generation pump cell including: a hydrogen generation electrode disposed on the oxygen-ion-conductive solid electrolyte layer in the reference gas chamber; and an outer electrode disposed at a position different from the reference gas chamber on the oxygen-ion-conductive solid electrolyte layer and corresponding to the hydrogen generation electrode; and
a detection electrode disposed on the proton-conductive solid electrolyte layer to be in contact with a measurement-object gas.

A proton-conductive solid electrolyte layer (or, proton conductor) is a solid material that has a property of conducting a proton (a hydrogen ion; H⁺ ion). An oxygen-ion-conductive solid electrolyte layer (or, oxygen-ion conductor) is a solid material that has a property of conducting an oxygen ion (O²⁻ion).

The control unit contained in the gas sensor of the present invention includes:
a reference gas adjusting part for adjusting a hydrogen concentration in the reference gas chamber by operating the hydrogen generation pump cell; and
a detecting part for detecting a target gas to be measured in a measurement-object gas.

### Embodiment 1

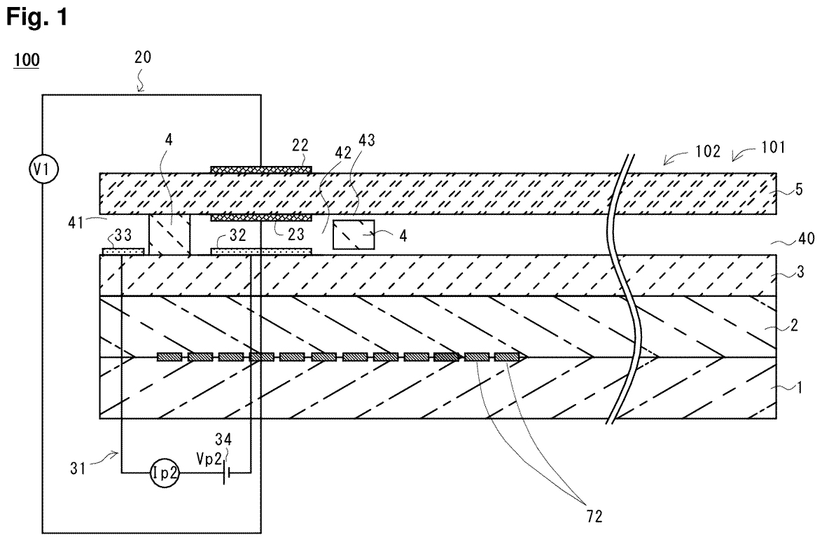

One example of embodiments of the gas sensor of the present invention will now be described with reference to the drawings. FIG. 1 is a vertical sectional schematic view in a longitudinal direction of a sensor element 101, showing one example of a schematic configuration of a gas sensor 100 of Embodiment 1. Hereinafter, based on FIG. 1, the upper side and the lower side in FIG. 1 are respectively defined as top and bottom, and the left side and the right side in FIG. 1 are respectively defined as a front end side and a rear end side. In FIG. 1, the gas sensor 100 represents one example of a gas sensor that detects hydrogen H₂ in a measurement-object gas by the sensor element 101, and measures the concentration of H₂.

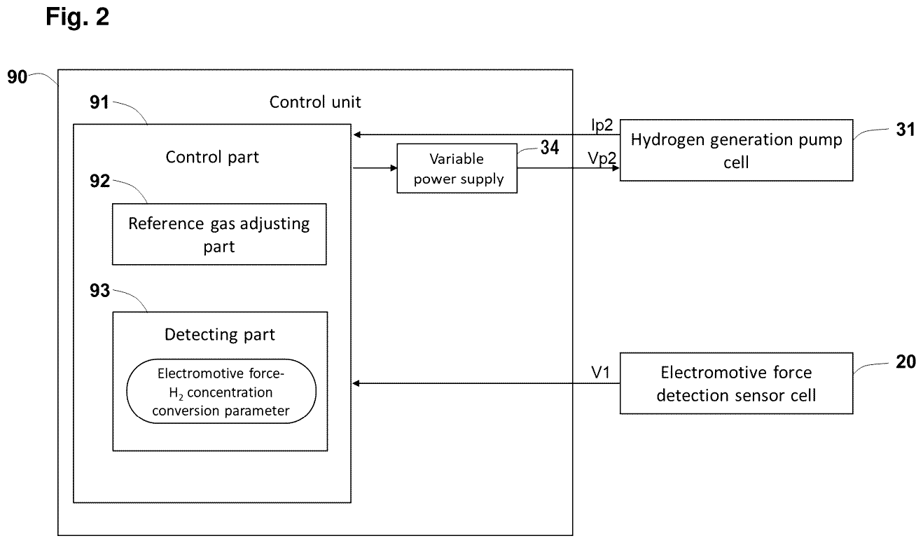

Further, the gas sensor 100 includes a control unit 90 for controlling the sensor element 101. FIG. 2 is a block diagram showing electric connections between the control unit 90 and the sensor element 101.

### Sensor Element

The sensor element 101 is an element in an elongated plate shape, including a base part 102 in an elongated plate shape having a proton-conductive solid electrolyte layer and an oxygen-ion-conductive solid electrolyte layer. The elongated plate shape also called a long plate shape or a belt shape.

The proton-conductive solid electrolyte layer is formed of a proton-conductive solid electrolyte (namely, a proton conductor), and extends in a longitudinal direction of the sensor element 101 (the base part 102). As the proton-conductive solid electrolyte (the proton conductor), for example, perovskite type oxide and the like may be used. As the proton-conductive solid electrolyte (the proton conductor), for example, a perovskite type ceramic represented by the following composition formula may be used.

A(BₓC₁₋ₓ)O_{3-δ}

Here, “A” is, for example, a bivalent metal selected from the group consisting of Ba, Ca, and Sr. “B” is, for example, a tetravalent metal selected from the group consisting of Ce and Zr. “C” is, for example, a trivalent metal selected from the group consisting of In, Y, Yb, Mn, and Sc. “C” is a so-called dopant. “x” may be 0 or more and 0.7 or less.

The oxygen-ion-conductive solid electrolyte layer is formed of an oxygen-ion-conductive solid electrolyte (namely, an oxygen-ion conductor), and extends in the longitudinal direction of the sensor element 101 (the base part 102). As the oxygen-ion-conductive solid electrolyte (the oxygen-ion conductor), for example, stabilized zirconia or partially stabilized zirconia, in which a rare earth metal oxide or an alkaline earth metal oxide is added to zirconia as a stabilizing agent, may be used. Examples of the stabilizing agents include yttria (Y₂O₃), calcia (CaO), magnesia (MgO), ceria (CeO₂), and scandia (Sc₂O₃). For example, yttria-stabilized zirconia may be used.

The base part 102 has such a structure that five layers, namely, a first substrate layer 1, a second substrate layer 2, an oxygen-ion conductor layer 3, a spacer layer 4, and a proton conductor layer 5, are layered in this order from the bottom side, as viewed in the drawing. Each of the first substrate layer 1 and the second substrate layer 2 is a layer formed of an insulator such as alumina. Each of the oxygen-ion conductor layer 3 and the spacer layer 4 is a layer formed of an oxygen-ion conductor, and is dense and gastight. The proton conductor layer 5 is a layer formed of a proton conductor, and is dense and gastight. These five layers all may have the same thickness, or the thickness may vary among the layers. The five layers are bonded together and integrated. The spacer layer 4 is a layer formed of the oxygen-ion conductor in Embodiment 1, but the present invention is not limited to this. The spacer layer 4 is required to be dense and gastight, and the spacer layer 4 may be a layer formed of a proton conductor or a layer formed of an insulator such as alumina.

The sensor element 101 is manufactured, for example, by stacking ceramic green sheets corresponding to the individual layers after conducting predetermined processing, printing of circuit pattern and the like, and then firing the stacked ceramic green sheets so that they are combined together.

An oxygen discharge space 41 is formed between a lower surface of the proton conductor layer 5 and an upper surface of the oxygen-ion conductor layer 3 in one end part in the longitudinal direction of the sensor element 101. Hereinafter, the one end part where the oxygen discharge space 41 exists is referred to as a front end part. The oxygen discharge space 41 is filled with a measurement-object gas.

A reference gas chamber 42 is formed between the proton-conductive solid electrolyte layer (namely, the proton conductor layer 5) and the oxygen-ion-conductive solid electrolyte layer (namely, the oxygen-ion conductor layer 3) inside the base part 102, at a position near the one end part (namely, the front end part) in the longitudinal direction of the sensor element 101 (or, the base part 102). That is, the reference gas chamber 42 is formed between the lower surface of the proton conductor layer 5 and the upper surface of the oxygen-ion conductor layer 3 at a position farther from the front end than the oxygen discharge space 41. The oxygen discharge space 41 and the reference gas chamber 42 are separated by the spacer layer 4 to prevent gas distribution between the oxygen discharge space 41 and the reference gas chamber 42.

In the longitudinal direction of the sensor element 101 (or, the base part 102), an air diffusion-rate limiting path 43 and an air introduction space 40 are formed to communicate in this order rearward the reference gas chamber 42. The air diffusion-rate limiting path 43 corresponds to an outside gas diffusion-rate limiting path of the present invention. The air introduction space 40 has an opening in the other end part (hereinafter, referred to as a rear end part) of the sensor element 101 (or, the base part 102).

The oxygen discharge space 41, the reference gas chamber 42, and the air introduction space 40 constitute internal spaces of the sensor element 101. Each of the internal spaces is provided in such a manner that a portion of the spacer layer 4 is hollowed out, and the top of each of the internal spaces is defined by the lower surface of the proton conductor layer 5, the bottom of each of the internal spaces is defined by the upper surface of the oxygen-ion conductor layer 3, and the lateral surface of each of the internal spaces is defined by the lateral surface of the spacer layer 4.

The air diffusion-rate limiting path 43 is provided as two laterally elongated slits (having the longitudinal direction of the openings in the direction perpendicular to the figure in FIG. 1). The air diffusion-rate limiting path 43 may be in such a form that a desired diffusion resistance is created, but the form is not limited to the slits.

The reference gas chamber 42 is a space where a reference gas that is reference for detection of hydrogen concentration. An outside gas such as the air is introduced into the reference gas chamber 42 from a space outside the sensor element 101 via the air introduction space 40 and the air diffusion-rate limiting path 43. By converting water vapor in the outside gas (in this embodiment, the air) introduced into the reference gas chamber 42 into hydrogen, the reference gas chamber 42 is filled with a reference gas containing hydrogen gas. Conversion of water vapor into hydrogen is performed by a hydrogen generation pump cell 31.

The hydrogen generation pump cell 31 includes a hydrogen generation electrode 32 disposed on the oxygen-ion-conductive solid electrolyte layer (the oxygen-ion conductor layer 3) in the reference gas chamber 42; and an outer electrode 33 disposed at a position different from the reference gas chamber 42 on the oxygen-ion-conductive solid electrolyte layer (the oxygen-ion conductor layer 3) and corresponding to the hydrogen generation electrode 32. The phrase “corresponding to the hydrogen generation electrode 32” means that the hydrogen generation electrode 32 and the outer electrode 33 are adjacent to each other via the oxygen-ion-conductive solid electrolyte.

That is, the hydrogen generation pump cell 31 is an electrochemical pump cell composed of the hydrogen generation electrode 32 disposed on the upper surface of the oxygen-ion conductor layer 3 in the reference gas chamber 42, the outer electrode 33 disposed on the upper surface of the oxygen-ion conductor layer 3 in the oxygen discharge space 41, and the oxygen-ion conductor layer 3 in contact with both the hydrogen generation electrode 32 and the outer electrode 33.

The hydrogen generation electrode 32 and the outer electrode 33 are porous cermet electrodes (electrodes in a state that a metal component and a ceramic component are mixed).

The ceramic component to be used is not particularly limited, but is preferably an oxygen-ion-conductive solid electrolyte as in the case of the oxygen-ion conductor layer 3. For example, ZrO₂ (stabilized ZrO₂) can be used as the ceramic component.

The hydrogen generation electrode 32 and the outer electrode 33 preferably contain a noble metal having catalytic activity (e.g., at least one of Pt, Rh, Ir, Ru, and Pd) as the metal component. For example, the hydrogen generation electrode 32 and the outer electrode 33 may be porous cermet electrodes made of Pt and ZrO₂.

The hydrogen generation electrode 32 functions also as a catalyst that decomposes water vapor H₂O in the outside gas (for example, the air) introduced into the reference gas chamber 42 through the air introduction space 40 and the air diffusion-rate limiting path 43.

In the hydrogen generation pump cell 31, a predetermined pump voltage Vp2 is applied between the hydrogen generation electrode 32 and the outer electrode 33 by a variable power supply 34 to make a pump current Ip2 flow between the hydrogen generation electrode 32 and the outer electrode 33, and thus water vapor H₂O in the reference gas chamber 42 is decomposed at the hydrogen generation electrode 32 (2H₂O→2H₂+O₂) to generate hydrogen H₂ and oxygen O₂. And, it is possible to pump out, from the reference gas chamber 42 to the oxygen discharge space 41, oxygen generated by decomposition of water vapor H₂O and oxygen originally contained in the outside gas introduced into the reference gas chamber 42.

By the operation of the hydrogen generation pump cell 31, hydrogen generated by decomposition of H₂O remains in the reference gas chamber 42 so that the reference gas chamber 42 is filled with the reference gas containing the hydrogen gas.

In this embodiment, the outer electrode 33 is disposed to be in contact with a measurement-object gas. The outer electrode 33 may be disposed at a position different from the reference gas chamber 42. The outer electrode 33 may be disposed to be in contact with the measurement-object gas as in the case of this embodiment. Alternatively, the outer electrode 33 may be disposed in the air introduction space 40 and in contact with the outside gas (namely, the air). The oxygen generated by the decomposition of H₂O in the outside gas introduced into the reference gas chamber 42 and the oxygen originally contained in the outside gas introduced into the reference gas chamber 42 are pumped out to the outer electrode 33. More preferably, the outer electrode 33 may be disposed to be in contact with the measurement-object gas as in the case of this embodiment. In other words, oxygen may be to be pumped out from the reference gas chamber 42 to the measurement-object gas. In this case, the pumped-out oxygen does not affect the outside gas to be introduced into the reference gas chamber 42, and it is therefore possible to operate the hydrogen generation pump cell 31 more effectively.

A hydrogen reference electrode 23 is disposed on the proton conductor layer 5 (on the lower surface of the proton conductor layer 5) in the reference gas chamber 42. A detection electrode 22 is disposed on a region of the upper surface of the proton conductor layer 5 that corresponds to the hydrogen reference electrode 23. The detection electrode 22 is disposed to be in contact with a measurement-object gas. In this embodiment, the detection electrode 22 is disposed on an outer surface of the sensor element 101. The gas sensor 100 is configured so that the measurement-object gas is present around the front end part of the sensor element 101, and the surroundings of the detection electrode 22 are in the measurement-object gas atmosphere.

The detection electrode 22, the hydrogen reference electrode 23, and the proton conductor layer 5 sandwiched between the detection electrode 22 and the hydrogen reference electrode 23 form an electrochemical sensor cell, namely, an electromotive force detection sensor cell 20. The hydrogen partial pressure (hydrogen concentration) in the measurement-object gas around the detection electrode 22 can be detected from an electromotive force V1 measured in the electromotive force detection sensor cell 20.

The detection electrode 22 and the hydrogen reference electrode 23 are porous cermet electrodes (electrodes in a state that a metal component and a ceramic component are mixed). The ceramic component to be used is not particularly limited, but is preferably a hydrogen-ion (proton) conductive solid electrolyte as in the case of the proton conductor layer 5. For example, strontium zirconate doped with yttrium (Y) can be used as the ceramic component.

The detection electrode 22 and the hydrogen reference electrode 23 preferably contain a noble metal having catalytic activity (e.g., at least one of Pt, Rh, Ir, Ru, and Pd) as the metal component. For example, the detection electrode 22 and the hydrogen reference electrode 23 may be porous cermet electrodes made of Pt and strontium zirconate doped with yttrium (Y).

The sensor element 101 further includes a heater 72 that functions as a temperature regulator of heating and maintaining the temperature of the sensor element 101 so as to enhance the hydrogen ion conductivity or the oxygen ion conductivity of the solid electrolytes.

The heater 72 is an electrical resistor sandwiched from top and bottom by the first substrate layer 1 and the second substrate layer 2 that are both composed of insulators. The heater 72 is connected with an external power supply via a lead wire not shown. The heater 72 is externally powered to generate heat, and heats and maintains the temperature of the solid electrolytes forming the sensor element 101.

The heater 72 is embedded over at least the whole area of the reference gas chamber 42 so that the temperature of the entire sensor element 101 can be adjusted to such a temperature that activates the solid electrolytes (both of the proton conductor layer 5 and the oxygen-ion conductor layer 3). The temperature may be adjusted so that the hydrogen generation pump cell 31 and the electromotive force detection sensor cell 20 are operable. It is not necessary that the whole area is adjusted to the same temperature, but the sensor element 101 may have temperature distribution. By maintaining the heater 72 at a desired temperature, the sensor element 101 can be maintained at a driving temperature at which the solid electrolyte is activated and thus hydrogen concentration is accurately measured. For example, the hydrogen generation pump cell 31 may be at about 700° C., and the electromotive force detection sensor cell 20 may be at about 600° C.

In the sensor element 101 of the present embodiment, the heater 72 is embedded in the base part 102, but this form is not limitative. The heater 72 may be disposed to heat the base part 102. That is, the heater 72 may heat the sensor element 101 to develop oxygen ion conductivity with which the hydrogen generation pump cell 31 is operable, and to develop hydrogen ion conductivity with which the electromotive force detection sensor cell 20 is operable. For example, the heater 72 may be embedded in the base part 102 in such a state that the heater 72 is sandwiched by the first substrate layer 1 and the second substrate layer 2 that are both composed of insulators, as in the present embodiment.

The first substrate layer 1 and the second substrate layer 2 need not be insulators, and may be a proton-conductive solid electrolyte as in the case of the proton conductor layer 5, or an oxygen-ion-conductive solid electrolyte layer as in the case of the oxygen-ion conductor layer 3. In this case, an insulating layer composed of an insulator such as alumina may be formed on the upper and lower surfaces of the heater 72 to ensure electrical insulation between and the heater 72 and the first substrate layer 1, and electrical insulation between the heater 72 and the second substrate layer 2. Alternatively, for example, a heater part may be formed as a heater substrate that is separate from the base part 102, and may be disposed at a position adjacent to the base part 102.

The above-described sensor element 101 is incorporated into the gas sensor 100 in such a form that the front end part of the sensor element 101 comes into contact with the measurement-object gas, and the rear end part of the sensor element 101 comes into contact with the outside gas such as the air.

### Control Unit

The gas sensor 100 of this embodiment includes the sensor element 101 described above and the control unit 90 for controlling the sensor element 101. In the gas sensor 100, each of the electrodes 22, 23, 32, and 33 of the sensor element 101 is electrically connected to the control unit 90 through a lead wire not shown. FIG. 2 is a block diagram showing electric connections between the control unit 90, and the hydrogen generation pump cell 31 and the electromotive force detection sensor cell 20 of the sensor element 101. The control unit 90 includes the above-described variable power supply 34 and a control part 91. The control part 91 includes a reference gas adjusting part 92 and a detecting part 93.

The control part 91 is realized by a general-purpose or dedicated computer, and functions as the reference gas adjusting part 92 and the detecting part 93 are realized by a CPU, a memory or the like installed in the computer. It is to be noted that when hydrogen in exhaust gas from an engine of a car is a target gas to be measured by the gas sensor 100 and the sensor element 101 is attached to an exhaust gas path, some or all of the functions of the control unit 90 (especially, the control unit 91) may be realized by an electronic control unit (ECU) installed in the car.

The control part 91 is configured to acquire an electromotive force V1 in the electromotive force detection sensor cell 20 of the sensor element 101. The control part 91 may be configured to further acquire a pump current Ip2 in the hydrogen generation pump cell 31. Further, the control part 91 is configured to output a control signal to the variable power supply 34.

The reference gas adjusting part 92 is configured to operate the hydrogen generation pump cell 31 so as to adjust a hydrogen concentration in a reference gas in the reference gas chamber 42.

In this embodiment, the reference gas adjusting part 92 is configured to adjust the hydrogen concentration in the reference gas chamber 42 by applying a predetermined voltage (the pump voltage Vp2) between the hydrogen generation electrode 32 and the outer electrode 33 of the hydrogen generation pump cell 31 to decompose water vapor H₂O in the outside gas introduced into the reference gas chamber 42 at the hydrogen generation electrode 32 (2H₂O→2H₂+O₂) so that hydrogen H₂ and oxygen O₂ are generated, and to pump out, from the reference gas chamber 42, the generated oxygen O₂ and an oxygen contained (namely, originally present) in the outside gas introduced into the reference gas chamber 42.

When a pump voltage Vp2 is applied between the hydrogen generation electrode 32 and the outer electrode 33 in the hydrogen generation pump cell 31 so that oxygen is pumped out from the reference gas chamber 42 to the external space (namely, oxygen discharge space 41), a pump current Ip2 increases as the pump voltage Vp2 is increased while the pump voltage Vp2 is low. At this time, oxygen gas present in the reference gas chamber 42 is pumped out. Subsequently, when the pump voltage Vp2 becomes high, the pump current Ip2 does not increase even when the pump voltage Vp2 is increased, and becomes to be saturated. A value of the saturated current at this time is referred to as a first limiting current value. A region in which the pump current Ip2 is at the first limiting current value with respect to the pump voltage Vp2 is referred to as a first limiting current region. In the first limiting current region, it is considered that substantially all of oxygen in the outside gas (in this embodiment, the air) introduced into the reference gas chamber 42 through the air diffusion-rate limiting path 43 is pumped out by the hydrogen generation pump cell 31. Therefore, the first limiting current value is to be a value corresponding to the oxygen concentration in the reference gas chamber 42. In this case, the pump current Ip2 flows from the hydrogen generation electrode 32 toward the outer electrode 33 in the outside of the sensor element 101.

When the pump voltage Vp2 becomes further high, the pump current Ip2 starts to increase again. This is because water vapor H₂O starts to be decomposed at the hydrogen generation electrode 32. That is, at the hydrogen generation electrode 32, water vapor H₂O is decomposed (2H₂O→2H₂+O₂) to generate hydrogen H₂ and oxygen O₂, and the generated oxygen O₂ is pumped out from the reference gas chamber 42. Subsequently, when the pump voltage Vp2 becomes further high, the pump current Ip2 does not increase even when the pump voltage Vp2 is increased, and becomes to be saturated again. A value of the saturated current at this time is referred to as a second limiting current value. A region in which the pump current Ip2 is at the second limiting current value with respect to the pump voltage Vp2 is referred to as a second limiting current region. In the second limiting current region, it is considered that substantially all of water vapor in the outside gas (in this embodiment, the air) introduced into the reference gas chamber 42 through the air diffusion-rate limiting path 43 is decomposed at the hydrogen generation electrode 32, and that substantially all of the oxygen generated by the decomposition of the water vapor is pumped out by the hydrogen generation pump cell 31. An amount of the oxygen generated by the decomposition of the water vapor is to be an amount corresponding to the water vapor concentration in the outside gas. Therefore, it is considered that the second limiting current value is to be the sum of the above-described first limiting current value corresponding to the oxygen concentration in the reference gas chamber 42, and a current value corresponding to the water vapor concentration in the reference gas chamber 42.

In driving the gas sensor 100, as described above, the reference gas adjusting part 92 applies the predetermined voltage (the pump voltage Vp2) between the hydrogen generation electrode 32 and the outer electrode 33 of the hydrogen generation pump cell 31 to decompose water vapor H₂O in the outside gas (in this embodiment, the air) introduced into the reference gas chamber 42. The pump voltage Vp2 may be set as a voltage such that the decomposition of the water vapor occurs at the hydrogen generation electrode 32. Preferably, the pump voltage Vp2 may be set as a voltage such that the pump current Ip2 is to be the above-described second limiting current. Alternatively, the pump voltage Vp2 may be set as a voltage such that the pump current Ip2 is to be at a predetermined current value, that is, a voltage such that a predetermined amount of oxygen is to be pumped out from the reference gas chamber 42. The pump voltage Vp2 may vary depending on the intended use of the gas sensor 100, the configuration of the sensor element 101 and the like, and the pump voltage Vp2 may be, for example, about 500 mV to 1500 mV.

When the pump voltage Vp2 is applied between the hydrogen generation electrode 32 and the outer electrode 33 of the hydrogen generation pump cell 31, substantially all of oxygen gas in the air introduced into the reference gas chamber 42 is pumped out to the oxygen discharge space 41. Further, water vapor in the air is decomposed to generate hydrogen and oxygen at the hydrogen generation electrode 32, and the generated oxygen is also pumped out from the reference gas chamber 42 to the oxygen discharge space 41. As a result, a reference gas containing hydrogen gas generated by the decomposition of the water vapor is to be present in the reference gas chamber 42.

The detecting part 93 is configured to detect a target gas to be measured (in this embodiment, hydrogen) in a measurement-object gas.

In this embodiment, the detecting part 93 is configured to detect the target gas to be measured (in this embodiment, hydrogen) in the measurement-object gas based on an electromotive force V1 between the detection electrode 22 and the hydrogen reference electrode 23 in the electromotive force detection sensor cell 20.

As described above, the detection electrode 22 is disposed on the outer surface of the sensor element 101 and in contact with the measurement-object gas. Also as described above, the reference gas containing the hydrogen gas generated by the decomposition of the water vapor is present in the reference gas chamber 42. In other words, the hydrogen reference electrode 23 is in contact with the reference gas containing the hydrogen gas generated by the decomposition of the water vapor.

The detecting part 93 may acquire the electromotive force V1 between the detection electrode 22 and the hydrogen reference electrode 23 in the electromotive force detection sensor cell 20, may calculate the H₂ concentration in the measurement-object gas on the basis of a previously-stored conversion parameter (electromotive force-H₂ concentration conversion parameter) between the electromotive force V1 and the H₂ concentration in the measurement-object gas, and may output the calculated H₂ concentration as a measurement value of the gas sensor 100. The electromotive force-H₂ concentration conversion parameter is previously stored in the memory of the control part 91 which functions as the detecting part 93. The electromotive force-H₂ concentration conversion parameter may appropriately be determined by those skilled in the art by, for example, previously performing an experiment on the gas sensor 100. The electromotive force-H₂ concentration conversion parameter may be, for example, the coefficient of an approximate expression (e.g., logarithmic function) obtained by experiment or a theoretical formula, or a map showing the relationship between the electromotive force V1 and the H₂ concentration in the measurement-object gas. The electromotive force-H₂ concentration conversion parameter may be specific to each individual gas sensor 100 or may be common to a plurality of gas sensors.

Consideration will be done on the assumption that the air itself is used as a reference gas as in the case of a conventional gas sensor. An amount of hydrogen gas containing the air is extremely small (about 5×10⁻⁸ % by volume), and concentration (partial pressure) of the hydrogen varies. FIG. 1 of the Non-Patent Document 2 discloses that a proton transport number of a proton-conductive solid electrolyte is much less than 1 at extremely low hydrogen partial pressure such as the air. It is known that when the proton transport number is 1, an electromotive force generated between a pair of electrodes disposed on the proton-conductive solid electrolyte follows the so-called Nernst equation. That is, an electromotive force is generated in accordance with difference (or, a ratio) between hydrogen partial pressure in a gas in contact with one electrode and hydrogen partial pressure in a gas in contact with the other electrode. However, the proton transport number is much less than 1 at extremely low hydrogen partial pressure such as the air. In such a case, electrode potential of a reference electrode in contact with the air is considered to deviate from a value derived from the Nernst equation. It is concerned that such a deviation of the electrode potential of the reference electrode may result in a decrease in the measurement accuracy of hydrogen concentration.

In order to address this concern, it is considered to use, as a reference gas, a gas with a predetermined hydrogen concentration such that the proton transport number is 1 or substantially 1. For example, a gas cylinder filled with a gas with a predetermined hydrogen concentration can be used to directly supply the gas with the predetermined hydrogen concentration to the sensor element 101. However, in this case, the gas sensor may become large, thereby being unsuitable for on-vehicle use or other applications where mounting space is limited.

On the other hand, in the present invention, the introduced air is adjusted to a reference gas containing hydrogen gas in the reference gas chamber 42, and the adjusted reference gas is used as the reference gas. Therefore, it is considered to be possible to maintain high measurement accuracy of hydrogen concentration. In addition, since a gas cylinder is not required and the gas sensor can be made compact, it is considered that the gas sensor can be used sufficiently for on-vehicle use or other applications.

Water vapor concentration in the air is not constant. Thus, an amount of hydrogen generated by decomposition of water vapor, that is, hydrogen concentration in the reference gas chamber 42 may vary. However, a range of the hydrogen concentration in the reference gas chamber 42 is considered to be sufficiently higher than a concentration of hydrogen originally contained in the air. Therefore, it is considered that the proton transport number is maintained at 1 or substantially 1. This is considered to make electrode potential of the hydrogen reference electrode 23 stable and maintain high measurement accuracy of the hydrogen concentration.

Further, the detecting part 93 may be configured to detect the target gas to be measured (in this embodiment, hydrogen) in the measurement-object gas based on the electromotive force V1 between the detection electrode 22 and the hydrogen reference electrode 23 in the electromotive force detection sensor cell 20, and the hydrogen concentration in the reference gas in the reference gas chamber 42. A hydrogen concentration in a measurement-object gas can be detected with further high accuracy, even if a hydrogen concentration in the reference gas chamber 42 varies.

For example, the detecting part 93 may acquire a water vapor concentration in the air, and may calculate the H₂ concentration in the reference gas in the reference gas chamber 42 on the basis of a previously-stored relationship between the pump voltage Vp2 applied to the hydrogen generation pump cell 31 and an amount of generated hydrogen at each of the water vapor concentrations. As the water vapor concentration in the air, for example, a value measured by a temperature and humidity meter or the like other than the gas sensor may be used. For example, when the reference gas adjusting part 92 applies a pump voltage Vp2 such that the pump current Ip2 is to be the above-described second limiting current, between the hydrogen generation electrode 32 and the outer electrode 33 of the hydrogen generation pump cell 31, H₂ concentration in the reference gas is considered to be substantially proportional to the water vapor concentration in the air.

The detecting part 93 may calculate the hydrogen concentration in the measurement-object gas based on the electromotive force V1 between the detection electrode 22 and the hydrogen reference electrode 23 in the electromotive force detection sensor cell 20, in consideration of the calculated H₂ concentration in the reference gas. For example, as the above-described conversion parameter (the electromotive force-H₂ concentration conversion parameter) between the electromotive force V1 and the H₂ concentration in the measurement-object gas, the detecting part 93 may store a map showing correspondence among the electromotive force V1, the H₂ concentration in the reference gas, and the H₂ concentration in the measurement-object gas.

### Embodiment 2

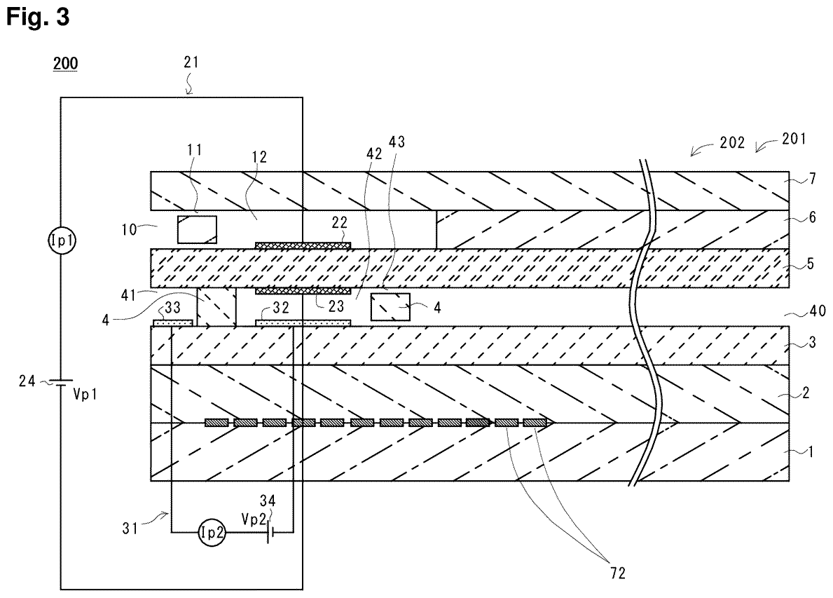

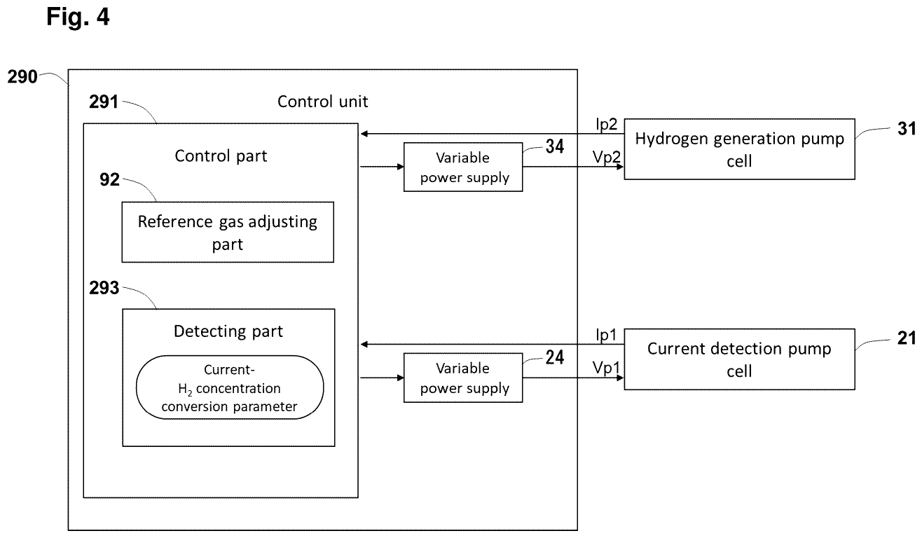

While an example of an electromotive force-type gas sensor that measures a hydrogen H₂ concentration in a measurement-object gas is described as Embodiment 1, the gas sensor of the present invention is not limited to this, and the gas sensor may be a limiting current-type gas senor. As a gas sensor 200 of Embodiment 2, one example of a limiting current-type gas sensor that measures a hydrogen H₂ concentration in a measurement-object gas is shown. FIG. 3 is a vertical sectional schematic view in the longitudinal direction of a sensor element 201, showing one example of a schematic configuration of the gas sensor 200 of Embodiment 2. In FIG. 3, the same member as in FIG. 1 is denoted by the same sign. FIG. 4 is a block diagram showing electric connections between a control unit 290 and the sensor element 201 in the gas sensor 200 of Embodiment 2.

In the sensor element 201, the base part 202 has such a structure that seven layers, namely, a first substrate layer 1, a second substrate layer 2, an oxygen-ion conductor layer 3, a spacer layer 4, a proton conductor layer 5, a second spacer layer 6, and a ceiling layer 7, are layered in this order from the bottom side, as viewed in the drawing. Each of the second spacer layer 6 and the ceiling layer 7 is a layer formed of an insulator such as alumina, as in the case of the first substrate layer 1 and the second substrate layer 2. These seven layers all may have the same thickness, or the thickness may vary among the layers. The seven layers are bonded together and integrated.

In the sensor element 201, a measurement-object gas cavity 12 into which a measurement-object gas is introduced via a measurement-object gas diffusion-rate limiting path 11 is formed inside the base part 202.

In the sensor element 201, a gas inlet 10 is formed between a lower surface of the ceiling layer 7 and an upper surface of the proton conductor layer 5 in one end part (namely, a front end part) in the longitudinal direction of the sensor element 201 (or, the base part 202). The measurement-object gas diffusion-rate limiting path 11 and the measurement-object gas cavity 12 are formed to communicate in this order in the longitudinal direction from the gas inlet 10.

The gas inlet 10, and the measurement-object gas cavity 12 constitute internal spaces of the sensor element 201. Each of the internal spaces is provided in such a manner that a portion of the second spacer layer 6 is hollowed out, and the top of each of the internal spaces is defined by the lower surface of the ceiling layer 7, the bottom of each of the internal spaces is defined by the upper surface of the proton conductor layer 5, and the lateral surface of each of the internal spaces is defined by the lateral surface of the second spacer layer 6.

The measurement-object gas diffusion-rate limiting path 11 is provided as two laterally elongated slits (having the longitudinal direction of the openings in the direction perpendicular to the figure in FIG. 3). The measurement-object gas diffusion-rate limiting path 11 may be in such a form that a desired diffusion resistance is created, but the form is not limited to the slits.

In the sensor element 201, a detection electrode 22 exists in the measurement-object gas cavity 12. That is, the detection electrode 22 is disposed on the upper surface of the proton conductor layer 5 in the measurement-object gas cavity 12.

The gas inlet 10 is open to an external space in which a measurement-object gas is present, and the measurement-object gas is taken into the sensor element 201 from the external space through the gas inlet 10.

The measurement-object gas diffusion-rate limiting path 11 creates a predetermined diffusion resistance to the measurement-object gas taken through the gas inlet 10.

The measurement-object gas cavity 12 is provided as a space for measuring a hydrogen concentration in the measurement-object gas introduced through the measurement-object gas diffusion-rate limiting path 11. The hydrogen concentration is measured by the operation of a current detection pump cell 21.

The current detection pump cell 21 includes the detection electrode 22 disposed on the proton-conductive solid electrolyte layer (the proton conductor layer 5) in the measurement-object gas cavity 12; and a hydrogen reference electrode 23 disposed at a position different from the measurement-object gas cavity 12 on the proton-conductive solid electrolyte layer (the proton conductor layer 5) and corresponding to the detection electrode 22. The phrase “corresponding to the detection electrode 22” means that the detection electrode 22 and the hydrogen reference electrode 23 are adjacent to each other via the proton-conductive solid electrolyte.

That is, the current detection pump cell 21 is an electrochemical pump cell composed of the detection electrode 22 disposed on the upper surface of the proton conductor layer 5 in the measurement-object gas cavity 12, the hydrogen reference electrode 23 disposed on the lower surface of the proton conductor layer 5 in the reference gas chamber 42, and the proton conductor layer 5 sandwiched between the detection electrode 22 and the hydrogen reference electrode 23.

In the current detection pump cell 21, a predetermined pump voltage Vp1 is applied between the detection electrode 22 and the hydrogen reference electrode 23 by a variable power supply 24 to make a pump current Ip1 flow between the detection electrode 22 and the hydrogen reference electrode 23, and thus it is possible to pump out hydrogen in the measurement-object gas cavity 12 to the reference gas chamber 42.

FIG. 4 is a block diagram showing electric connections between the control unit 290 and the respective pump cells 21 and 31 of the sensor element 201 in the gas sensor 200 of Embodiment 2. In FIG. 4, the same member as in FIG. 2 is denoted by the same sign. The control unit 290 includes the variable power supplies 24 and 34, and a control part 291. The control part 291 includes a reference gas adjusting part 92 and a detecting part 293.

The control part 291 is configured to acquire a pump current Ip1 in the current detection pump cell 21 of the sensor element 201. The control part 91 may be configured to further acquire a pump current Ip2 in the hydrogen generation pump cell 31. Further, the control part 291 is configured to output control signals to the variable power supplies 24 and 34.

In the gas sensor 200, the detecting part 293 is configured to detect a target gas to be measured (in Embodiment 2, hydrogen) in a measurement-object gas based on a current (pump current Ip1) flowing between the detection electrode 22 and the hydrogen reference electrode 23 in the current detection pump cell 21.

When a pump voltage Vp1 is applied between the detection electrode 22 and the hydrogen reference electrode 23 of the current detection pump cell 21 so that hydrogen is pumped out from the measurement-object gas cavity 12 to the reference gas chamber 42, a pump current Ip1 increases as the pump voltage Vp1 is increased while the pump voltage Vp1 is low. At this time, hydrogen gas present in the measurement-object gas cavity 12 is pumped out. Subsequently, when the pump voltage Vp1 becomes high, the pump current Ip1 does not increase even when the pump voltage Vp1 is increased, and becomes to be saturated. A value of the saturated current at this time is referred to as a limiting current value of hydrogen gas. A region in which the pump current Ip1 is at the limiting current value of the hydrogen gas with respect to the pump voltage Vp1 is referred to as a limiting current region of hydrogen gas. In the limiting current region of the hydrogen gas, it is considered that substantially all of hydrogen in the measurement-object gas introduced into the measurement-object gas cavity 12 through the measurement-object gas diffusion-rate limiting path 11 is pumped out by the current detection pump cell 21. In this case, the pump current Ip1 flows from the hydrogen reference electrode 23 toward the detection electrode 22 in the outside of the sensor element 201.

In driving the gas sensor 200, the detecting part 293 applies a predetermined voltage (the pump voltage Vp1) between the detection electrode 22 and the hydrogen reference electrode 23 of the current detection pump cell 21 to pump out hydrogen in the measurement-object gas introduced into the measurement-object gas cavity 12 from the measurement-object gas cavity 12, and detects the pump current Ip1 flowing at the time.

The pump voltage Vp1 may be set as a voltage such that the pump current Ip1 is to be the above-described limiting current of the hydrogen gas. Thus, substantially all of hydrogen in the measurement-object gas introduced into the measurement-object gas cavity 12 is pumped out. In this case, the pump current Ip1 flowing through the current detection pump cell 21 is to be a current value corresponding to the hydrogen concentration in the measurement-object gas. Therefore, the hydrogen concentration in the measurement-object gas can be detected based on the pump current Ip1. The pump voltage Vp1 may vary depending on the intended use of the gas sensor 200, the configuration of the sensor element 201 and the like, and the pump voltage Vp1 may be, for example, about 400 mV to 1000 mV.

The detecting part 293 may acquire the pump current Ip1 flowing between the detection electrode 22 and the hydrogen reference electrode 23 in the current detection pump cell 21, may calculate H₂ concentration in a measurement-object gas on the basis of a previously-stored conversion parameter (current-H₂ concentration conversion parameter) between the pump current Ip1 and the H₂ concentration in the measurement-object gas, and may output the H₂ concentration as a measurement value of the gas sensor 200. The current-H₂ concentration conversion parameter is previously stored in the memory of the control part 291 which functions as the detecting part 293. The current-H₂ concentration conversion parameter may appropriately be determined by those skilled in the art by, for example, previously performing an experiment on the gas sensor 200. The current-H₂ concentration conversion parameter may be, for example, the coefficient of an approximate expression (e.g., linear function) obtained by experiment or a theoretical formula, or a map showing the relationship between the pump current Ip1 and the H₂ concentration in a measurement-object gas. The current-H₂ concentration conversion parameter may be specific to each individual gas sensor 200 or may be common to a plurality of gas sensors.

In the gas sensor 200, hydrogen concentration in the reference gas chamber 42 is adjusted by the reference gas adjusting part 92, and thus electrode potential of the hydrogen reference electrode 23 is stable. Therefore, in the current detection pump cell 21, it is considered that a relationship between the applied pump voltage Vp1 and the pump current Ip1 flowing through the current detection pump cell 21 better corresponds to hydrogen concentration in the measurement-object gas, and that the hydrogen concentration can therefore be measured more accurately.

### Embodiment 3

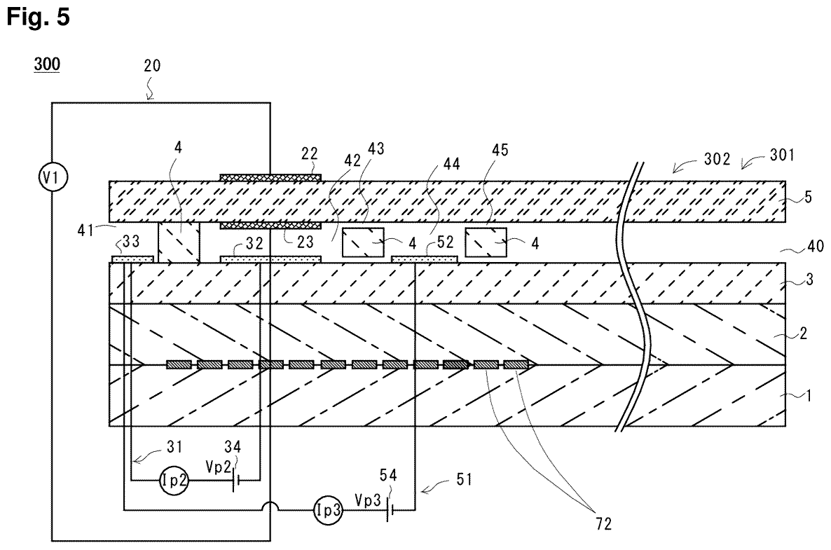

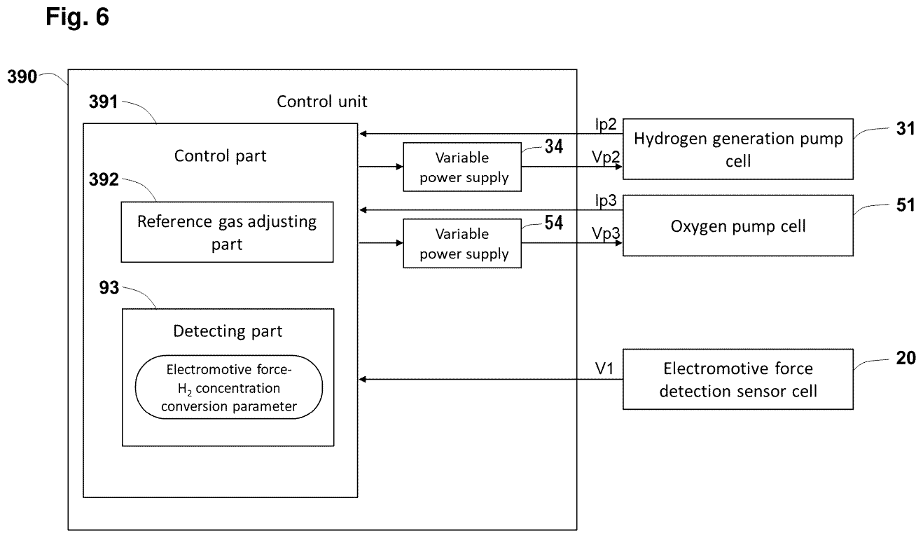

In the present invention, as described above, a reference gas containing hydrogen is present in the reference gas chamber 42. Another example of the configuration of the reference gas chamber 42 and its surroundings will be shown. As a gas sensor 300 of Embodiment 3, another example of the electromotive force-type gas sensor that measures a hydrogen H₂ concentration in a measurement-object gas is shown. FIG. 5 is a vertical sectional schematic view in the longitudinal direction of a sensor element 301, showing one example of a schematic configuration of the gas sensor 300 of Embodiment 3. In FIG. 5, the same member as in FIG. 1 is denoted by the same sign. FIG. 6 is a block diagram showing electric connections between a control unit 390 and the sensor element 301 in the gas sensor 300 of Embodiment 3.

In the sensor element 301, a pretreatment chamber 44 is formed between the proton-conductive solid electrolyte layer (the proton conductor layer 5) and the oxygen-ion-conductive solid electrolyte layer (the oxygen-ion conductor layer 3) inside a base part 302 and adjacent to the reference gas chamber 42 via the outside gas diffusion-rate limiting path (the air diffusion-rate limiting path 43), into the pretreatment chamber the outside gas (in this embodiment, the air) being introduced via a pretreatment diffusion-rate limiting path 45.

The pretreatment chamber 44 constitutes an internal space of the sensor element 301. The internal space is provided, as in the case of the reference gas chamber 42, in such a manner that a portion of the spacer layer 4 is hollowed out, and the top of the internal space is defined by the lower surface of the proton conductor layer 5, the bottom of the internal space is defined by the upper surface of the oxygen-ion conductor layer 3, and the lateral surface of the internal space is defined by the lateral surface of the spacer layer 4.

The pretreatment diffusion-rate limiting path 45 is provided as two laterally elongated slits (having the longitudinal direction of the openings in the direction perpendicular to the figure in FIG. 5), as in the case of the air diffusion-rate limiting path 43. The pretreatment diffusion-rate limiting path 45 may be in such a form that a desired diffusion resistance is created, but the form is not limited to the slits.

The pretreatment chamber 44 is provided as a space for previously pumping out oxygen in an outside gas (in this embodiment, the air) introduced through the air introduction space 40 and the pretreatment diffusion-rate limiting path 45. The oxygen in the air is pumped out by an oxygen pump cell 51.

The oxygen pump cell 51 includes an oxygen pump electrode 52 disposed on the oxygen-ion-conductive solid electrolyte layer (the oxygen-ion conductor layer 3) in the pretreatment chamber 44; and an outer electrode disposed at a position different from the reference gas chamber 42 and the pretreatment chamber 44 on the oxygen-ion-conductive solid electrolyte layer (the oxygen-ion conductor layer 3) and corresponding to the oxygen pump electrode 52. The phrase “corresponding to the oxygen pump electrode 52” means that the oxygen pump electrode 52 and the outer electrode are adjacent to each other via the oxygen-ion-conductive solid electrolyte.

That is, the oxygen pump cell 51 is an electrochemical pump cell composed of the oxygen pump electrode 52 disposed on the upper surface of the oxygen-ion conductor layer 3 in the pretreatment chamber 44, the outer electrode 33 disposed on the upper surface of the oxygen-ion conductor layer 3 in the oxygen discharge space 41, and the oxygen-ion conductor layer 3 in contact with both the oxygen pump electrode 52 and the outer electrode 33.

In Embodiment 3, the outer electrode 33 functions also as the outer electrode of the oxygen pump cell 51. The outer electrode of the oxygen pump cell 51, and the outer electrode of the hydrogen generation pump cell 31 may be formed as different electrodes, or may be formed as one electrode as in this embodiment.

The oxygen pump electrode 52 is, as in the case of the hydrogen generation electrode 32 and the outer electrode 33, a porous cermet electrode (an electrode in a state that a metal component and a ceramic component are mixed). The ceramic component to be used is not particularly limited, but is preferably an oxygen-ion-conductive solid electrolyte as in the case of the oxygen-ion conductor layer 3. For example, ZrO₂ (stabilized ZrO₂) can be used as the ceramic component.

The oxygen pump electrode 52 preferably contains, as in the case of the hydrogen generation electrode 32 and the outer electrode 33, a noble metal having catalytic activity (e.g., at least one of Pt, Rh, Ir, Ru, and Pd) as the metal component. For example, the oxygen pump electrode 52 may be a porous cermet electrode made of Pt and ZrO₂.

In the oxygen pump cell 51, a predetermined pump voltage Vp3 is applied between the oxygen pump electrode 52 and the outer electrode 33 by a variable power supply 54 to make a pump current Ip3 flow between the oxygen pump electrode 52 and the outer electrode 33, and thus it is possible to pump out oxygen in the pretreatment chamber 44 to the oxygen discharge space 41. The outside gas (in this embodiment, the air) after oxygen has been pumped out by the operation of the oxygen pump cell 51 is introduced into reference gas chamber 42 via the air diffusion-rate limiting path 43.

FIG. 6 is a block diagram showing electric connections between the control unit 390, and the hydrogen generation pump cell 31, the oxygen pump cell 51, and the electromotive force detection sensor cell 20 of the sensor element 301 in the gas sensor 300 of Embodiment 3. In FIG. 6, the same member as in FIG. 2 is denoted by the same sign. The control unit 390 includes the variable power supplies 34 and 54, and a control part 391. The control part 391 includes a reference gas adjusting part 392 and a detecting part 93.

The control part 391 is configured to acquire an electromotive force V1 in the electromotive force detection sensor cell 20 of the sensor element 301. The control part 391 may be configured to further acquire a pump current (Ip2, Ip3) in each of the respective pump cells 31 and 51. Further, the control part 391 is configured to output control signals to the variable power supplies 34 and 54.

In the gas sensor 300, the reference gas adjusting part 392 is configured to adjust the hydrogen concentration in the reference gas chamber 42 by operating the oxygen pump cell 51 to pump out oxygen in an outside gas (in this embodiment, the air) introduced into the pretreatment chamber 44; and operating the hydrogen generation pump cell 31 to decompose water vapor in the outside gas introduced into the reference gas chamber 42 after the oxygen in the outside gas is pumped out in the pretreatment chamber 44 so that hydrogen and oxygen are generated, and to pump out, from the reference gas chamber 42, the generated oxygen and the oxygen originally contained in the outside gas introduced into the reference gas chamber 42.

More specifically, in the gas sensor 300, the reference gas adjusting part 392 applies a predetermined voltage (the pump voltage Vp3) between the oxygen pump electrode 52 and the outer electrode 33 of the oxygen pump cell 51 to pump out at least a part of oxygen in the air introduced into the pretreatment chamber 44. And, for the air introduced into the reference gas chamber 42 after the oxygen is pumped out in the pretreatment chamber 44, the reference gas adjusting part 392 is configured to adjust hydrogen concentration in the reference gas chamber 42 by applying a predetermined voltage (the pump voltage Vp2) between the hydrogen generation electrode 32 and the outer electrode 33 of the hydrogen generation pump cell 31 to decompose water vapor H₂O in said air at the hydrogen generation electrode 32 (2H₂O→2H₂+O₂) so that hydrogen H₂ and oxygen O₂ are generated, and to pump out the generated oxygen O₂ and an oxygen O₂ remained in said air from the reference gas chamber 42.

When a pump voltage Vp3 is applied between the oxygen pump electrode 52 and the outer electrode 33 of the oxygen pump cell 51 so that oxygen is pumped out from the pretreatment chamber 44 to the external space (namely, the oxygen discharge space 41), a pump current Ip3 increases as the pump voltage Vp3 is increased while the pump voltage Vp3 is low. At this time, oxygen gas present in the pretreatment chamber 44 is pumped out. Subsequently, when the pump voltage Vp3 becomes high, the pump current Ip3 does not increase even when the pump voltage Vp3 is increased, and becomes to be saturated. A value of the saturated current at this time is referred to as a limiting current value of oxygen gas. A region in which the pump current Ip3 is at the limiting current value of the oxygen gas with respect to the pump voltage Vp3 is referred to as a limiting current region of oxygen gas. In the limiting current region of the oxygen gas, it is considered that substantially all of oxygen in the outside gas (in this embodiment, the air) introduced into the pretreatment chamber 44 through the pretreatment diffusion-rate limiting path 45 is pumped out by the oxygen pump cell 51. In this case, the pump current Ip3 flows from the oxygen pump electrode 52 toward the outer electrode 33 in the outside of the sensor element 101.

In driving the gas sensor 300, as described above, the reference gas adjusting part 392 applies the predetermined voltage (the pump voltage Vp3) between the oxygen pump electrode 52 and the outer electrode 33 of the oxygen pump cell 51 to pump out at least a part of oxygen in the outside gas (in this embodiment, the air). The pump voltage Vp3 may be set as a voltage such that at least a part of oxygen in the outside gas is pumped out by the oxygen pump cell 51. Preferably, the pump voltage Vp3 may be set as a voltage such that most of the oxygen in the outside gas is pumped out by the oxygen pump cell 51. More preferably, the pump voltage Vp3 may be set as a voltage such that the pump current Ip3 is to be at the above-described limiting current value of the oxygen gas. Further, the pump voltage Vp3 may be set as a voltage such that water vapor in the air is not decomposed. The pump voltage Vp3 may vary depending on the intended use of the gas sensor 300, the configuration of the sensor element 301 and the like, and the pump voltage Vp3 may be, for example, about 100 mV to 400 mV. The pump voltage Vp3 may be, for example, about 200 mV to 300 mV.

In the gas sensor 300, the reference gas adjusting part 392 previously pumps out, by the oxygen pump cell 51, at least a part of oxygen in the outside gas (in this embodiment, the air) introduced into the pretreatment chamber 44. Then, the air after at least the part of the oxygen has been pumped out by the oxygen pump cell 51 (that is, the air whose oxygen concentration has been adjusted to a low level) is introduced into the reference gas chamber 42. In the reference gas chamber 42, the reference gas adjusting part 392 decomposes water vapor in the air whose oxygen concentration has been adjusted to the low level by the hydrogen generation pump cell 31 to generate hydrogen and oxygen. The reference gas adjusting part 392 pumps out the generated oxygen and oxygen (residual oxygen) contained in the air by the hydrogen generation pump cell 31 to adjust a hydrogen concentration in the reference gas in the reference gas chamber 42. Thus, a reference gas containing hydrogen gas generated by the decomposition of the water vapor is to be present in the reference gas chamber 42.

In the gas sensor 100 of Embodiment 1 described above, the hydrogen generation pump cell 31 has two functions, namely, a function of pumping out oxygen in the air, and a function of decomposing water vapor in the air and pumping out the generated oxygen. On the other hand, in the gas sensor 300 of Embodiment 3, the reference gas adjusting part 392 is configured to previously pump out oxygen in the air by the oxygen pump cell 51. In other words, the oxygen pump cell 51 has a function of pumping out oxygen in the air, and the hydrogen generation pump cell 31 mainly has a function of decomposing water vapor in the air and pumping out the generated oxygen. As a result, in the gas sensor 300 of Embodiment 3, the pump current Ip2 to flow through the hydrogen generation pump cell 31 becomes smaller in comparison with the case of the gas sensor 100 of Embodiment 1. Therefore, pumping ability of the hydrogen generation pump cell 31 has more margin, and thus ability of decomposing water vapor can be maintained at a higher level. As a result, the hydrogen concentration in the reference gas can be precisely adjusted. Further, even if the hydrogen generation electrode 32 gradually deteriorates due to long-time use, the accuracy of the hydrogen concentration in the reference gas can be maintained.

### Embodiment 4

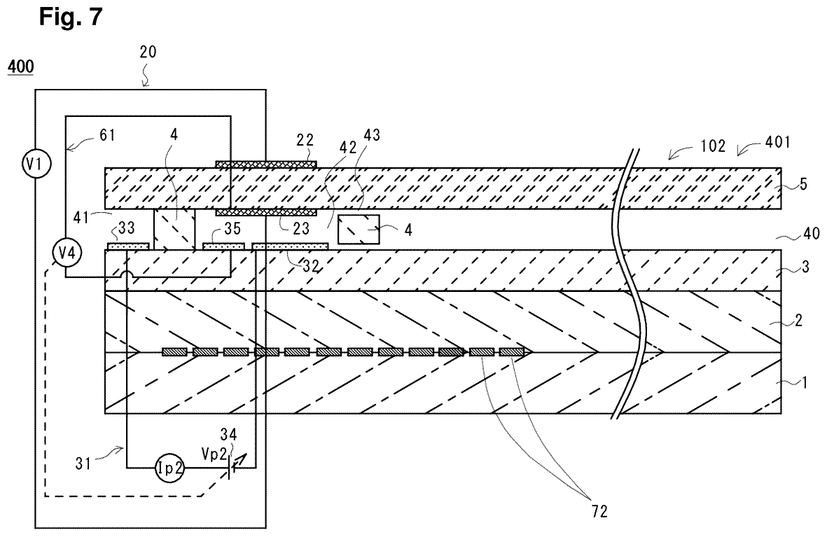

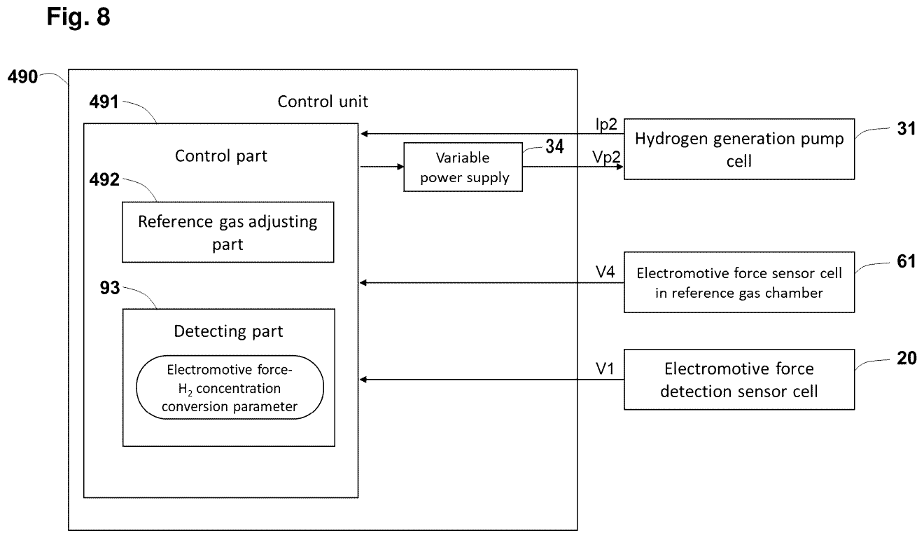

As a gas sensor 400 of Embodiment 4, another example of the electromotive force-type gas sensor that measures a hydrogen H₂ concentration in a measurement-object gas is shown. FIG. 7 is a vertical sectional schematic view in the longitudinal direction of a sensor element 401, showing one example of a schematic configuration of the gas sensor 400 of Embodiment 4. In FIG. 7, the same member as in FIG. 1 is denoted by the same sign. FIG. 8 is a block diagram showing electric connections between a control unit 490 and the sensor element 401 in the gas sensor 400 of Embodiment 4.

An oxygen reference electrode 35 may further be disposed on the oxygen-ion-conductive solid electrolyte layer (the oxygen-ion conductor layer 3) in the reference gas chamber 42.

In the sensor element 401, the oxygen reference electrode 35 is disposed at a position farther from the outside gas diffusion-rate limiting path (namely, the air diffusion-rate limiting path 43) than the hydrogen generation electrode 32 on the oxygen-ion-conductive solid electrolyte layer (the oxygen-ion conductor layer 3) in the reference gas chamber 42.

That is, the oxygen reference electrode 35 is disposed at a position closer to the one end part (the front end part) in the longitudinal direction of the sensor element 401 (the base part 102) than the hydrogen generation electrode 32 on the upper surface of the oxygen-ion conductor layer 3 in the reference gas chamber 42.

The oxygen reference electrode 35 is, as in the case of the hydrogen generation electrode 32 and the outer electrode 33, a porous cermet electrode (an electrode in a state that a metal component and a ceramic component are mixed). The ceramic component to be used is not particularly limited, but is preferably an oxygen-ion-conductive solid electrolyte as in the case of the oxygen-ion conductor layer 3. For example, ZrO₂ (stabilized ZrO₂) can be used as the ceramic component.

The oxygen reference electrode 35 preferably contains, as in the case of the hydrogen generation electrode 32 and the outer electrode 33, a noble metal having catalytic activity (e.g., at least one of Pt, Rh, Ir, Ru, and Pd) as the metal component. For example, the oxygen reference electrode 35 may be a porous cermet electrode made of Pt and ZrO₂.

The hydrogen reference electrode 23, the oxygen reference electrode 35, and the proton conductor layer 5, the spacer layer 4 and the oxygen-ion conductor layer 3 that are present between the hydrogen reference electrode 23 and the oxygen reference electrode 35 form an electrochemical sensor cell, namely, an electromotive force detection sensor cell 61 in the reference gas chamber. The water vapor partial pressure (water vapor concentration) in the reference gas chamber 42 can be detected from an electromotive force V4 measured in the electromotive force detection sensor cell 61 in the reference gas chamber.

In the electromotive force detection sensor cell 61 in the reference gas chamber, the proton conductive solid electrolyte (the proton conductor layer 5) and the oxygen ion conductive solid electrolyte (the spacer layer 4 and the oxygen-ion conductor layer 3) are joined (or, bonded). The hydrogen reference electrode 23 on the proton conductive solid electrolyte (the proton conductor layer 5), and the oxygen reference electrode 35 on the oxygen ion conductive solid electrolyte (the spacer layer 4 and the oxygen-ion conductor layer 3) are both present in the reference gas chamber 42. That is, the hydrogen reference electrode 23 on the proton conductive solid electrolyte (the proton conductor layer 5), and the oxygen reference electrode 35 on the oxygen ion conductive solid electrolyte (the spacer layer 4 and the oxygen-ion conductor layer 3) are both in contact with the same gas atmosphere. Referring to JP 2022-110596 A, in the electromotive force detection sensor cell 61 in the reference gas chamber having such a configuration, an electromotive force is considered to be generated in accordance with water vapor partial pressure (water vapor concentration) in the reference gas chamber 42.

FIG. 8 is a block diagram showing electric connections between the control unit 490, and the hydrogen generation pump cell 31, the electromotive force detection sensor cell 61 in the reference gas chamber and the electromotive force detection sensor cell 20 of the sensor element 401 in the gas sensor 400 of Embodiment 4. In FIG. 8, the same member as in FIG. 2 is denoted by the same sign. The control unit 490 includes the variable power supply 34 and a control part 491. The control part 491 includes a reference gas adjusting part 492 and a detecting part 93.

The control part 491 is configured to acquire an electromotive force (V1, V4) in each of the respective sensor cell 20 and 61 of the sensor element 401. The control part 491 may be configured to further acquire a pump current Ip2 in the hydrogen generation pump cell 31. Further, the control part 491 is configured to output a control signal to the variable power supply 34.

In the gas sensor 400, the reference gas adjusting part 492 may be configured to operate the hydrogen generation pump cell 31 based on the electromotive force V4 between the hydrogen reference electrode 23 and the oxygen reference electrode 35 in the electromotive force detection sensor cell 61 in the reference gas chamber.

More specifically, in the gas sensor 400, the reference gas adjusting part 492 performs feedback control of the pump voltage Vp2 of the variable power supply 34 in the hydrogen generation pump cell 31 so that the electromotive force V4 between the hydrogen reference electrode 23 and the oxygen reference electrode 35 in the electromotive force detection sensor cell 61 in the reference gas chamber is at a predetermined value (referred to as a set value V4_{SET}). The reference gas adjusting part 492 applies the pump voltage Vp2 in the hydrogen generation pump cell 31 to decompose water vapor H₂O in the outside gas introduced into the reference gas chamber 42 at the hydrogen generation electrode 32 (2H₂O→2H₂ +O₂) so that hydrogen H₂ and oxygen O₂ are generated, and to pump out the generated oxygen O₂ and an oxygen O₂ originally contained in the outside gas introduced into the reference gas chamber 42 from the reference gas chamber 42, thereby adjusting a hydrogen concentration in the reference gas chamber 42. The set value V4_{SET} may be set as a value such that substantially all of water vapor H₂O in the outside gas introduced into the reference gas chamber 42 is decomposed at the hydrogen generation electrode 32. By setting the set value V4_{SET} in such a manner, water vapor H₂O may be decomposed with higher accuracy, and it may therefore be possible to adjust the hydrogen concentration in the reference gas chamber 42 with higher accuracy. The set value V4_{SET} may vary depending on the intended use of the gas sensor 400, the configuration of the sensor element 401 and the like, and the set value V4_{SET} may be, for example, about 500 mV to 1500 mV.

Further, in the gas sensor 400, the detecting part 93 may also be configured to detect the target gas to be measured (in this embodiment, hydrogen) in the measurement-object gas based on the electromotive force V1 between the detection electrode 22 and the hydrogen reference electrode 23 in the electromotive force detection sensor cell 20, and the hydrogen concentration in the reference gas in the reference gas chamber 42. A hydrogen concentration in a measurement-object gas can be detected with further high accuracy, even if a hydrogen concentration in the reference gas chamber 42 varies.

For example, the detecting part 93 may acquire a water vapor concentration in the air, may calculate a concentration of residual water vapor (an amount of residual water vapor) in the reference gas chamber 42 from the electromotive force V4 in the electromotive force detection sensor cell 61 in the reference gas chamber, and may calculate an amount (an amount of decomposed water vapor) of water vapor decomposed in the reference gas chamber 42 from difference between the acquired water vapor concentration in the air and the calculated concentration of the residual water vapor in the reference gas chamber 42. Since the amount of the decomposed water vapor corresponds to an amount of the generated hydrogen, the detecting part 93 may calculate H₂ concentration in the reference gas in the reference gas chamber 42 based on the amount of the decomposed water vapor. As the water vapor concentration in the air, for example, a value measured by a temperature and humidity meter or the like other than the gas sensor may be used.

The detecting part 93 may calculate the hydrogen concentration in the measurement-object gas based on the electromotive force V1 between the detection electrode 22 and the hydrogen reference electrode 23 in the electromotive force detection sensor cell 20, in consideration of the calculated H₂ concentration in the reference gas. For example, as the above-described conversion parameter (the electromotive force-H₂ concentration conversion parameter) between the electromotive force V1 and the H₂ concentration in the measurement-object gas, the detecting part 93 may previously store a map showing correspondence among the electromotive force V1, the H₂ concentration in the reference gas, and the H₂ concentration in the measurement-object gas.

In the above-described sensor element 401, the oxygen reference electrode 35 is disposed at a position farther from the air diffusion-rate limiting path 43 than the hydrogen generation electrode 32 on the oxygen-ion conductor layer 3 in the reference gas chamber 42. That is, on the oxygen-ion conductor layer 3 in the reference gas chamber 42, the oxygen reference electrode 35 and the hydrogen generation electrode 32 are disposed in series in this order from a side near to the front end part in the longitudinal direction of the sensor element 401. However, the position of the oxygen reference electrode 35 is not limited to this.

The oxygen reference electrode 35 may be disposed on the oxygen-ion conductor layer 3 in the reference gas chamber 42. The oxygen reference electrode 35 may be disposed at a position closer to the air diffusion-rate limiting path 43 than the hydrogen generation electrode 32. That is, on the oxygen-ion conductor layer 3 in the reference gas chamber 42, the hydrogen generation electrode 32 and the oxygen reference electrode 35 may be disposed in series in this order from the side near to the front end part in the longitudinal direction of the sensor element 401. Alternatively, the oxygen reference electrode 35 and the hydrogen generation electrode 32 may be disposed in parallel in the longitudinal direction of the sensor element 401.

In the above-described sensor element 401, the oxygen reference electrode 35 and the hydrogen generation electrode 32 are disposed as separate electrodes, but the oxygen reference electrode 35 and the hydrogen generation electrode 32 may be disposed as an integrated electrode. That is, the integrated electrode may be served as both the oxygen reference electrode 35 and the hydrogen generation electrode 32. In this case, the integrated electrode, the outer electrode 33 and the oxygen-ion conductor layer 3 may constitute the hydrogen generation pump cell 31, and the integrated electrode, the hydrogen reference electrode 23, the oxygen-ion conductor layer 3, the spacer layer 4 and the proton conductor layer 5 may constitute the electromotive force detection sensor cell 61 in the reference gas chamber.

Referring to the above JP 2022-110596 A, in order to detect the electromotive force V4 in accordance with a water vapor concentration in the reference gas chamber 42, in the electromotive force detection sensor cell 61 in the reference gas chamber, it is required that the proton conductive solid electrolyte and the oxygen ion conductive solid electrolyte are joined (or, bonded). The spacer layer 4 present between the proton conductor layer 5 and the oxygen-ion conductor layer 3 may be an oxygen ion conductive solid electrolyte layer as in the case of the sensor element 401. In this case, the proton conductive solid electrolyte and the oxygen ion conductive solid electrolyte are joined (or, bonded) between the lower surface of the proton conductor layer 5 and the upper surface of the spacer layer 4. Alternatively, the spacer layer 4 may be a proton conductive solid electrolyte layer. In this case, the proton conductive solid electrolyte and the oxygen ion conductive solid electrolyte are joined (or, bonded) between the lower surface of the spacer layer 4 and the upper surface of the oxygen-ion conductor layer 3. A joint surface (or, bonded surface) between the proton conductive solid electrolyte and the oxygen ion conductive solid electrolyte may exist inside the spacer layer 4. The whole of the spacer layer 4 need not be the proton conductive solid electrolyte and/or the oxygen ion conductive solid electrolyte. It is sufficient that the proton conductive solid electrolyte and the oxygen ion conductive solid electrolyte are joined (or, bonded) by at least a part of the spacer layer 4.

Embodiments 1 to 4 have been described above as examples of the embodiments according to the present invention, but the present invention is not limited thereto. The present invention may include a gas sensor having any structure including a sensor element and a control unit as long as the object of the present invention can be achieved, that is, a gas sensor that can measures hydrogen gas or a gas containing a hydrogen atom (such as ammonia NH₃, water vapor H₂O, and hydrocarbon HC) in a measurement-object gas with higher accuracy is provided.

The above Embodiments 1 to 4 show examples of gas sensors that measure a hydrogen concentration in a measurement-object gas, but a target gas to be measured is not limited to hydrogen. Examples of the target gases to be measured other than hydrogen H₂ include ammonia NH₃, water vapor H₂O, and hydrocarbon HC. Examples of hydrocarbon HC include alkane such as methane (e.g. methane CH₄, ethane C₂H₆, propane C₃H₈, and butane C₄H₁₀), and alkene such as ethylene (e.g. ethylene C₂H₄, propylene C₃H₆, and butylene C₄H₈). That is, a gas sensor of the present invention can measure hydrogen gas or a gas containing a hydrogen atom (such as ammonia NH₃, water vapor H₂O, and hydrocarbon HC) in a measurement-object gas.

In the case of measuring a gas containing a hydrogen atom (such as ammonia NH₃, water vapor H₂O, and hydrocarbon HC), for example, the sensor element 201 shown in FIG. 3 can be used. For example, when measuring ammonia NH₃ as a gas containing a hydrogen atom, the detection electrode 22 functions also as a catalyst that decomposes ammonia NH₃ in the measurement-object gas introduced into the measurement-object gas cavity 12 through the measurement-object gas diffusion-rate limiting path 11.

The detecting part 293 may apply a predetermined voltage (pump voltage Vp1) between the detection electrode 22 and the hydrogen reference electrode 23 of the current detection pump cell 21 to decompose ammonia NH₃ in the measurement-object gas introduced into the measurement-object gas cavity 12 at the detection electrode 22, and to pump out hydrogen generated by the decomposition from the measurement-object gas cavity 12 by the current detection pump cell 21. Ammonia NH₃ may be measured by detecting the pump current Ip1 flowing at the time.

The pump voltage Vp1 may be set as a value such that substantially all of ammonia NH₃ in the measurement-object gas introduced into the measurement-object gas cavity 12 is decomposed. In this case, the pump current Ip1 flowing through the current detection pump cell 21 is to be a current value corresponding to the concentration of ammonia NH₃ in the measurement-object gas. Therefore, the concentration of ammonia NH₃ in the measurement-object gas can be detected based on the pump current Ip1. A concentration of a gas containing a hydrogen atom other than ammonia NH₃ (e.g. water vapor H₂O, alkane such as methane CH₄, and alkene such as ethylene C₂H₄) can be detected in the same manner. In the case of measuring a gas containing a hydrogen atom, the pump voltage Vp1 may vary depending on the target gas species, the intended use of the gas sensor 200, the configuration of the sensor element 201 and the like, and the pump voltage Vp1 may be, for example, about 800 mV to 1200 mV.

In the above Embodiments 1 to 4, the detection electrode 22 is disposed on the region of the upper surface of the proton conductor layer 5 that corresponds to the hydrogen reference electrode 23. However, the present invention is not limited thereto. The detection electrode 22 may be disposed on the proton conductor layer 5 to be in contact with a measurement-object gas. For example, the detection electrode 22 may be disposed at a position different from the hydrogen reference electrode 23 in the longitudinal direction of the sensor element on the proton conductor layer 5. The detection electrode 22 may be disposed on, for example, the lower surface of the proton conductor layer 5 in the oxygen discharge space 41. Alternately, the detection electrode 22 may be disposed on a side surface or a front end surface of the proton conductor layer 5.

Both of the gas sensor 300 of Embodiment 3 including the oxygen pump cell 51, and the gas sensor 400 of Embodiment 4 including the electromotive force detection sensor cell 61 in the reference gas chamber are examples of the electromotive force-type gas sensors. In the electromotive force-type gas sensor, both of the oxygen pump cell 51 and the electromotive force detection sensor cell 61 in the reference gas chamber may be used. In the limiting current-type gas sensor, the oxygen pump cell 51 and/or the electromotive force detection sensor cell 61 in the reference gas chamber may be used.

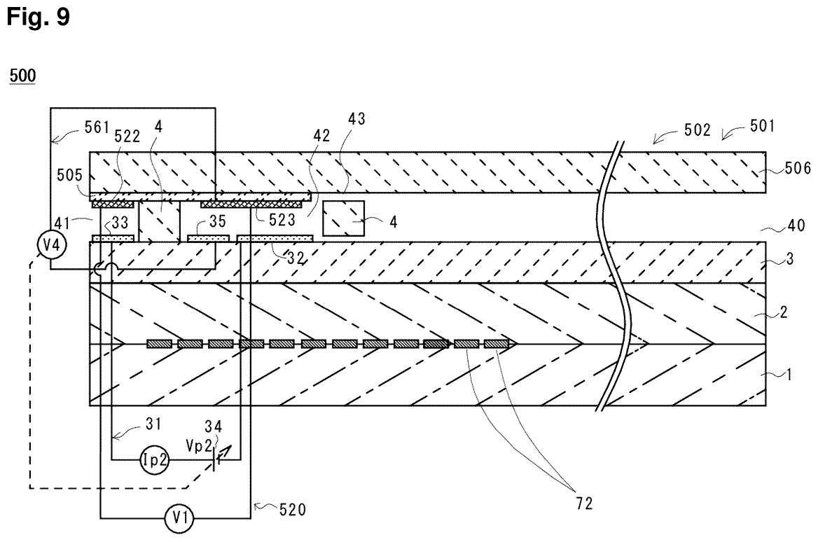

In the above Embodiments 1 to 4, both of the proton conductor layer 5 and the oxygen-ion conductor layer 3 are layers that extend over the entire length in the longitudinal direction of the sensor element, but the layer configuration is not limited to this. For example, the proton conductor layer and/or the oxygen-ion conductor layer may be present at a part of the entire length of the sensor element on which each of electrodes is to be disposed. FIG. 9 is a vertical sectional schematic view in the longitudinal direction of a sensor element 501, showing one example of a schematic configuration of the gas sensor 500 of Embodiment 5. The gas sensor 500 of Embodiment 5 is a variation with respect to the gas sensor 400 of Embodiment 4, in which the layer configuration of the sensor element is different. In FIG. 9, the same member as in FIG. 7 is denoted by the same sign.

In the sensor element 501, the base part 502 has such a structure that six layers, namely, a first substrate layer 1, a second substrate layer 2, an oxygen-ion conductor layer 3, a spacer layer 4, a proton conductor layer 505, and a second oxygen-ion conductor layer 506, are layered in this order from the bottom side, as viewed in the drawing. Each of the first substrate layer 1, the second substrate layer 2, the oxygen-ion conductor layer 3, the spacer layer 4, and the second oxygen-ion conductor layer 506 is a layer that extends over the entire length in the longitudinal direction of the base part 502. On the other hand, the proton conductor layer 505 is present at a position from the front end part in the longitudinal direction of the sensor element 501 to substantially the entire surface in the reference gas chamber 42, between the lower surface of the second oxygen-ion conductor layer 506 and the upper surface of the spacer layer 4. The second oxygen-ion conductor layer 506 is a layer formed of an oxygen-ion conductor as in the case of the oxygen-ion conductor layer 3. Each of the proton conductor layer 505 and the second oxygen-ion conductor layer 506 is dense and gastight.

In the sensor element 501, a hydrogen reference electrode 523 is disposed on the proton conductor layer 505 (on the lower surface of the proton conductor layer 505) in the reference gas chamber 42. A detection electrode 522 is disposed on the proton conductor layer 505 (on the lower surface of the proton conductor layer 505) in the oxygen discharge space 41. The oxygen discharge space 41 is, as described above, filled with a measurement-object gas. The detection electrode 522, the hydrogen reference electrode 523, and the proton conductor layer 5 in contact with both the detection electrode 522 and the hydrogen reference electrode 523 form an electrochemical sensor cell, namely, an electromotive force detection sensor cell 520. The hydrogen partial pressure (hydrogen concentration) in the measurement-object gas around the detection electrode 522 can be detected from an electromotive force V1 measured in the electromotive force detection sensor cell 520.

The hydrogen reference electrode 523, the oxygen reference electrode 35, and the proton conductor layer 505, the spacer layer 4 and the oxygen-ion conductor layer 3 that are present between the hydrogen reference electrode 523 and the oxygen reference electrode 35 form an electrochemical sensor cell, namely, an electromotive force detection sensor cell 561 in the reference gas chamber. The water vapor partial pressure (water vapor concentration) in the reference gas chamber 42 can be detected from an electromotive force V4 measured in the electromotive force detection sensor cell 561 in the reference gas chamber.

The gas sensor 500 of Embodiment 5 can measure hydrogen in the measurement-object gas in the same manner as the gas sensor 400 of Embodiment 4. In the gas sensor 500 of Embodiment 5, the proton conductor layer 505 is shorter in length in the longitudinal direction of the sensor element 501 and thinner in thickness compared with other layers. In manufacturing the sensor element 501, for example, a ceramics green sheet manufactured by tape casting can be used as a sheet to be each of the first substrate layer 1, the second substrate layer 2, the oxygen-ion conductor layer 3, the spacer layer 4, and the second oxygen-ion conductor layer 506. For the proton conductor layer 505, a ceramics green sheet manufactured by tape casting may be used as in the case of other layers. Alternatively, the proton conductor layer 505 may be formed on a layer to be the second oxygen-ion conductor layer 506 by other method such as screen printing. When the proton conductor layer 505 is formed by other method, it is possible to reduce the kind of layers to be prepared by the tape casting. Further, this can have a positive impact on productivity, such as reduction in consumption of the proton conductor.

In the sensor element 501, the oxygen ion conductor is used as a base, and the proton conductor is arranged on a region where the detection electrode 522 and the hydrogen reference electrode 523 are to be disposed. However, the present invention is not limited to this. In contract, the proton conductor may be used as a base, and the oxygen ion conductor may be arranged on a region where the hydrogen generation electrode 32, the outer electrode 33, and the oxygen reference electrode 35 are to be disposed. Alternatively, the insulator such as alumina may be used as a base, and the proton conductor and the oxygen ion conductor may be arranged on a region where respective electrodes are to be disposed. While the gas sensor 500 of Embodiment 5 has a configuration corresponds to the gas sensor 400 of Embodiment 4, also as in the case of Embodiment 1 to 3, the proton conductor layer and/or the oxygen-ion conductor layer may be present at a part of the entire length of the sensor element on which each of electrodes is to be disposed.

Further, the present invention includes a gas adjusting device described below.

A gas adjusting device comprising:
a gas chamber at least partially surrounded by a proton-conductive solid electrolyte layer and an oxygen-ion-conductive solid electrolyte layer, into the gas chamber an outside gas being introduced via an outside gas diffusion-rate limiting path;
a hydrogen generation pump cell including: a hydrogen generation electrode disposed on the oxygen-ion-conductive solid electrolyte layer in the gas chamber; and an outer electrode disposed at a position different from the gas chamber on the oxygen-ion-conductive solid electrolyte layer and corresponding to the hydrogen generation electrode;
a hydrogen reference electrode disposed on the proton-conductive solid electrolyte layer in the gas chamber; and
a gas adjusting part for adjusting a hydrogen concentration in the gas chamber by operating the hydrogen generation pump cell. Here, the gas adjusting part may adjust the hydrogen concentration in the gas chamber by applying a predetermined voltage between the hydrogen generation electrode and the outer electrode of the hydrogen generation pump cell to decompose water vapor in the outside gas introduced into the gas chamber at the hydrogen generation electrode so that hydrogen and oxygen are generated, and to pump out the generated oxygen and an oxygen contained in the outside gas from the gas chamber; and supplies the gas whose hydrogen concentration is adjusted to the hydrogen reference electrode.

Configuration of the above-described gas adjusting device appears in FIG. 1 and FIG. 2. The reference gas chamber 42 in FIG. 1 corresponds to the gas chamber in the gas adjusting device, and the reference gas adjusting part 92 in FIG. 2 corresponds to the gas adjusting part in the gas adjusting device. The components and their functions in the gas adjusting device are as described in the above Embodiment 1.

### EXPLANATION OF REFERENCE SIGNS IN THE DRAWINGS

1: first substrate layer; 2: second substrate layer; 3: oxygen-ion conductor layer; 4: spacer layer; 5, 505: proton conductor layer; 506: second oxygen-ion conductor layer; 6: second spacer layer; 7: ceiling layer; 10: gas inlet; 11: measurement-object gas diffusion-rate limiting path; 12: measurement-object gas cavity; 20, 520: electromotive force detection sensor cell; 21: current detection pump cell; 22: detection electrode; 23: hydrogen reference electrode; 24: variable power supply (of the current detection pump cell); 31: hydrogen generation pump cell; 32: hydrogen generation electrode; 33: outer electrode; 34: variable power supply (of the hydrogen generation pump cell); 35: oxygen reference electrode; 40: air introduction space; 41: oxygen discharge space; 42: reference gas chamber; 43: air diffusion-rate limiting path; 44: pretreatment chamber; 45: pretreatment diffusion-rate limiting path 51: oxygen pump cell; 52: oxygen pump electrode; 54: variable power supply (of the oxygen pump cell); 61, 561: electromotive force detection sensor cell in the reference gas chamber; 72: heater; 90, 290, 390, 490: control unit; 91, 291, 391, 491: control part; 92, 392, 492: reference gas adjusting part; 93, 293: detecting part; 100, 200, 300, 400, 500: gas sensor; 101, 201, 301, 401, 501: sensor element; and 102, 202, 302, 502: base part.

What is claimed is:

1. A gas sensor for detecting a target gas to be measured in a measurement-object gas, the gas sensor comprising a sensor element and a control unit for controlling the sensor element, wherein
the sensor element comprises:
a base part in an elongated plate shape, including a proton-conductive solid electrolyte layer and an oxygen-ion-conductive solid electrolyte layer;
a reference gas chamber formed between the proton-conductive solid electrolyte layer and the oxygen-ion-conductive solid electrolyte layer inside the base part, into the reference gas chamber an outside gas being introduced via an outside gas diffusion-rate limiting path;
a hydrogen reference electrode disposed on the proton-conductive solid electrolyte layer in the reference gas chamber;
a hydrogen generation pump cell including: a hydrogen generation electrode disposed on the oxygen-ion-conductive solid electrolyte layer in the reference gas chamber; and an outer electrode disposed at a position different from the reference gas chamber on the oxygen-ion-conductive solid electrolyte layer and corresponding to the hydrogen generation electrode; and
a detection electrode disposed on the proton-conductive solid electrolyte layer to be in contact with a measurement-object gas; and
the control unit comprises:
a reference gas adjusting part for adjusting a hydrogen concentration in the reference gas chamber by operating the hydrogen generation pump cell; and
a detecting part for detecting a target gas to be measured in a measurement-object gas.

2. The gas sensor according to claim 1, wherein the reference gas adjusting part adjusts the hydrogen concentration in a reference gas in the reference gas chamber by applying a predetermined voltage between the hydrogen generation electrode and the outer electrode of the hydrogen generation pump cell to decompose water vapor in the outside gas introduced into the reference gas chamber at the hydrogen generation electrode so that hydrogen and oxygen are generated, and to pump out the generated oxygen and an oxygen contained in the outside gas from the reference gas chamber.

3. The gas sensor according to claim 1, wherein the detecting part detects the target gas to be measured in the measurement-object gas based on an electromotive force between the detection electrode and the hydrogen reference electrode.

4. The gas sensor according to claim 1, wherein the sensor element further comprises a measurement-object gas cavity formed inside the base part, into the measurement-object gas cavity the measurement-object gas being introduced via a measurement-object gas diffusion-rate limiting path,
the detection electrode exits in the measurement-object gas cavity, and
the detecting part detects the target gas to be measured in the measurement-object gas based on a current flowing between the detection electrode and the hydrogen reference electrode.

5. The gas sensor according to claim 1, wherein the sensor element comprises:
a pretreatment chamber formed between the proton-conductive solid electrolyte layer and the oxygen-ion-conductive solid electrolyte layer inside the base part and adjacent to the reference gas chamber via the outside gas diffusion-rate limiting path, into the pretreatment chamber the outside gas being introduced via a pretreatment diffusion-rate limiting path; and
an oxygen pump cell including: an oxygen pump electrode disposed on the oxygen-ion-conductive solid electrolyte layer in the pretreatment chamber; and an outer electrode disposed at a position different from the reference gas chamber and the pretreatment chamber on the oxygen-ion-conductive solid electrolyte layer and corresponding to the oxygen pump electrode, and
the reference gas adjusting part adjusts the hydrogen concentration in the reference gas chamber by operating the oxygen pump cell to pump out oxygen in the outside gas introduced into the pretreatment chamber; and operating the hydrogen generation pump cell to decompose water vapor in the outside gas introduced into the reference gas chamber after the oxygen in the outside gas is pumped out in the pretreatment chamber so that hydrogen and oxygen are generated, and to pump out the generated oxygen and the oxygen contained in the outside gas from the reference gas chamber.

6. The gas sensor according to claim 1, wherein the sensor element further comprises an oxygen reference electrode disposed on the oxygen-ion-conductive solid electrolyte layer in the reference gas chamber, and
the reference gas adjusting part operates the hydrogen generation pump cell based on an electromotive force between the hydrogen reference electrode and the oxygen reference electrode.

7. The gas sensor according to claim 1, wherein the outer electrode of the hydrogen generation pump cell is disposed to be in contact with the measurement-object gas.

8. The gas sensor according to claim 1, wherein the target gas to be measured is hydrogen, ammonia, water vapor, or methane.

9. A sensor element for detecting a target gas to be measured in a measurement-object gas, the sensor element comprising:
a base part in an elongated plate shape, including a proton-conductive solid electrolyte layer and an oxygen-ion-conductive solid electrolyte layer;
a reference gas chamber formed between the proton-conductive solid electrolyte layer and the oxygen-ion-conductive solid electrolyte layer inside the base part, into the reference gas chamber an outside gas being introduced via an outside gas diffusion-rate limiting path;
a hydrogen reference electrode disposed on the proton-conductive solid electrolyte layer in the reference gas chamber;
a hydrogen generation pump cell including: a hydrogen generation electrode disposed on the oxygen-ion-conductive solid electrolyte layer in the reference gas chamber; and an outer electrode disposed at a position different from the reference gas chamber on the oxygen-ion-conductive solid electrolyte layer and corresponding to the hydrogen generation electrode; and
a detection electrode disposed on the proton-conductive solid electrolyte layer to be in contact with a measurement-object gas.

10. A gas adjusting device comprising:
a gas chamber at least partially surrounded by a proton-conductive solid electrolyte layer and an oxygen-ion-conductive solid electrolyte layer, into the gas chamber an outside gas being introduced via an outside gas diffusion-rate limiting path;
a hydrogen generation pump cell including: a hydrogen generation electrode disposed on the oxygen-ion-conductive solid electrolyte layer in the gas chamber; and an outer electrode disposed at a position different from the gas chamber on the oxygen-ion-conductive solid electrolyte layer and corresponding to the hydrogen generation electrode;
a hydrogen reference electrode disposed on the proton-conductive solid electrolyte layer in the gas chamber; and
a gas adjusting part for adjusting a hydrogen concentration in the gas chamber by operating the hydrogen generation pump cell.
